(19) United States
(12) Patent Application Publication
(10) Pub. No.: US 2008/0085511 A1
(43) Pub. Date: Apr. 10, 2008

(54) Preparation of biopolymer arrays

(76) Inventors: Bill J. Peck, Mountain View, CA (US); Eric M. Leproust, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/543,369

(22) Filed: Oct. 5, 2006

(51) Int. Cl. C12Q 1/68; B41J 2/01; G06F 19/00; C12M 3/00

(52) U.S. Cl. 435/6; 435/287.2; 427/2.11; 702/20; 347/1

(57) ABSTRACT

Methods and apparatus are disclosed for preparing an array of biopolymer features on a substrate by initiating multiple cycles of drop deposition of biopolymer forming reagents to feature locations on the surface of the substrate. Each cycle comprises at least one step of deposition of biopolymer monomers or biopolymer subunits. For at least one of the biopolymer forming reagents and for at least a portion of the method, a dispensing protocol is employed wherein the biopolymer forming reagent is dispensed from a separate drop dispenser of a drop dispensing device and each drop dispenser comprises multiple nozzles. Multiple passes of the drop dispensing devices are employed for each cycle. In each cycle of the dispensing protocol drops of reagents are dispensed to feature locations from at least two different nozzles for each reagent in at least two different passes of a drop dispensing device. The cycles are repeated a sufficient number of times to prepare the array of biopolymer features.

### BACKGROUND OF THE INVENTION

This invention relates in general to methods of preparing arrays of features on a substrate. In some embodiments the invention relates to the manufacture of substrates having bound to the surfaces thereof a plurality of chemical compounds, such as biopolymers. In some embodiments, the invention relates to the manufacture of microarray slides comprising biopolymer features using drop dispensing devices.

In the field of diagnostics and therapeutics, it is often useful to attach species to a surface. One important application is in solid phase chemical synthesis wherein initial derivatization of a substrate surface enables synthesis of polymers such as oligonucleotides and peptides on the substrate itself. Substrate bound oligomer arrays, particularly oligonucleotide arrays, may be used in screening studies for determination of binding affinity.

The arrays may be microarrays created on the surface of a substrate by in situ synthesis of biopolymers such as polynucleotides, which include oligonucleotides, polypeptides, which include oligopeptides, polysaccharides, which include oligosaccharides, etc., and combinations thereof, or by deposition of molecules such as oligonucleotides, cDNA and so forth. Oligonucleotide arrays may be utilized to conduct multiplex analysis of multiple variant sites in one or more different target polynucleotides at the same time. Substrate bound oligomer arrays, particularly oligonucleotide arrays, may be used in screening studies for determination of binding affinity.

The deposition methods basically involve depositing biopolymer reagents at predetermined locations on a substrate, which are suitably activated such that the biopolymer reagents can link thereto. In some approaches biopolymer monomer reagents are deposited to different locations to form biopolymers of different sequence. In some approaches biopolymer reagents of different sequence may be deposited at different regions of the substrate to yield the completed array. Washing or other additional steps may also be used.

Similar technologies can be used for in situ synthesis of biopolymer arrays, such as DNA oligomer arrays, on a solid substrate. In this case, each oligomer is formed nucleotide by nucleotide directly in the desired location on the substrate surface. This process demands repeatable drop size and accurate placement on the substrate.

The in situ synthesis methods include those described in U.S. Pat. No. 5,449,754 for synthesizing peptide arrays, as well as WO 98/41531 and the references cited therein for synthesizing polynucleotides (specifically, DNA). Such in situ synthesis methods can be basically regarded as repeating at each spot the sequence of: (a) deprotecting any previously deposited monomer so that it can now link with a subsequently deposited protected monomer; and (b) depositing a droplet of another protected monomer for linking. Different monomers may be deposited at different regions on the substrate during any one iteration or cycle so that the different regions of the completed array will have different desired biopolymer sequences. One or more intermediate further steps may be required in each iteration or cycle, such as oxidation, capping and washing steps.

As indicated above, one of the steps in the synthesis process usually involves depositing small volumes or microdroplets of liquid containing reagents for the synthesis, for example, monomeric subunits or whole polynucleotides, onto to a surface of a support or substrate. In one approach, pulse-jet techniques are employed in depositing small volumes of liquid for synthesis of chemical compounds on the surface of substrates. For example, arrays may be fabricated by depositing droplets from a pulse-jet in accordance with known techniques. Pulse-jets include piezo jets and thermal jets. Given the above requirements of biopolymer array fabrication, deposition using pulse-jet techniques is particularly favorable. In particular, pulse-jet deposition has advantages that include producing very small spot sizes. Furthermore, the spot size is uniform and reproducible. Since it is a non-contact technique, pulse-jet deposition does not result in scratching or damaging the surface of the support on which the arrays are synthesized. Pulsejet techniques have very high deposition rate, which facilitates rapid manufacture of arrays. Conventional array deposition systems, including in situ systems, usually involve a single pulsejet that deposits all of a particular nucleoside monomer unit during a cycle,

In array fabrication, the quantities of polynucleotide available are usually very small and expensive. Additionally, sample quantities available for testing are usually also very small and it is therefore desirable to simultaneously test the same sample against a large number of different probes on an array. These conditions require use of arrays with large numbers of very small, closely spaced features. It is important in such arrays that features actually be present, that they are put down accurately in the desired target pattern, are of the correct size, and that the DNA is uniformly coated within the feature. Failure to meet such quality requirements can have serious consequences to diagnostic, screening, gene expression analysis or other purposes for which the array is being used.

In conventional array fabrication, there may be instances where one or more repeatable or non-repeatable errors occur during the deposition of reagents on the surface of a support. In the case of both the deposition of previously obtained biopolymers, but particularly in the in situ fabrication method, drop deposition errors from cycle to cycle may be different and are cumulative in determining errors in the finally formed features. Such errors may result, for example, from non-delivery of reagent from one or more of dispensing elements such as the dispensing nozzles of an inkjet or pulse jet apparatus. Errors of this kind basically render the support non-usable because one or more of the biopolymers deposited on the surface are incorrect.

In instances of error occurrence, the particular synthesis is stopped, the support is discarded, the source of the error, e.g., clogged printing nozzle, is fixed and a new synthesis is carried out with a new support starting from the beginning of the synthetic procedure. As may be appreciated, the occurrence of an error gives rise to a considerable amount of lost time, material and reagents.

It is also known to employ error detection systems to detect errors that occur during an array fabrication utilizing drop dispensing devices. When an error is detected, a different nozzle of a dispensing element is employed to redeposit the missing reagent. For example, if a pulsejet fails to fire during a single cycle at a feature location, the resulting feature will effectively be useless (since it will be capped in the capping step or, where no capping step is used, will be missing a nucleotide and therefore will have the wrong sequence). It is known to use multiple firings of a same reagent from a same pulsejet, during a same cycle. While this reduces random errors that might occur during a pulsejet firing, it does not correct for a fixed trajectory error of a pulsejet, nor will it correct for failure of that pulsejet. It is also known to employ a method wherein the reagent drop set deposited during one or more cycles for one or more of the multiple addresses includes, for a corresponding cycle and address, drops of a same reagent which are deposited from different deposition units during the same cycle.

It would be desirable then to provide a means by which errors in features formed during an in situ or any array fabrication method can be reduced. It would further be desirable to reduce errors that occur as a result of a drop dispensing element (such as a pulse jet of a multiple pulse jet system) particularly where one or more nozzles of a drop dispensing element become momentarily or permanently inoperable.

### SUMMARY

In some embodiments the present invention is directed to methods for preparing an array of biopolymer features on a substrate by initiating multiple cycles of drop deposition of biopolymer forming reagents to feature locations on the surface of the substrate. Each cycle comprises at least one step of deposition of biopolymer monomers or biopolymer subunits. For at least one of the biopolymer forming reagents and for at least a portion of the method, a dispensing protocol is employed wherein the biopolymer forming reagent is dispensed from a separate drop dispenser of a drop dispensing device and each drop dispenser comprises multiple nozzles. Multiple passes of the drop dispensing devices are employed for each cycle. In each cycle of the dispensing protocol drops of reagents are dispensed to feature locations from at least two different nozzles for each reagent in at least two different passes of a drop dispensing device. The cycles are repeated a sufficient number of times to prepare the array of biopolymer features.

In some embodiments of the above method, the two different nozzles are on the same drop dispenser. In some embodiments of the above method, the two different nozzles are on two different drop dispensers.

In some embodiments the biopolymer features are polynucleotide features and the biopolymer forming reagents comprises nucleotide monomers and activators. In some embodiments at least the nucleotide monomers and activator are dispensed for the entire method according to the above protocol until the array of biopolymers is prepared. In some embodiments at least the activator is dispensed according to the above protocol for the entire method until the array of biopolymers is prepared. In some embodiments at least the nucleotide monomers and activator are dispensed for a portion of the method until the array of biopolymers is patterned and the preparation of the array is completed by any suitable protocol.

In some embodiments the invention is directed to methods for preparing an array of polynucleotide features on a substrate by initiating multiple cycles of drop deposition of polynucleotide forming reagents to feature locations on the surface of the substrate. Each cycle comprises at least one step of deposition of polynucleotide monomers or polynucleotide subunits. For at least one of the polynucleotide forming reagents and for at least a portion of the method, a dispensing protocol is employed wherein the polynucleotide forming reagent is dispensed from a separate drop dispenser of a drop dispensing device and each drop dispenser comprises multiple nozzles. Multiple passes of the drop dispensing devices are employed for each cycle. In each cycle of the dispensing protocol drops of reagents are dispensed to feature locations from at least two different nozzles for each reagent in at least two different passes of a drop dispensing device. The cycles are repeated a sufficient number of times to prepare the array of biopolymer features.

In some embodiments of the above method, the two different nozzles are on the same drop dispenser. In some embodiments of the above method, the two different nozzles are on two different drop dispensers.

In some embodiments the polynucleotide forming reagents comprise nucleotide monomers and activators. In some embodiments at least the nucleotide monomers and activator are dispensed according to the above protocol for the entire method until the array of biopolymers is prepared. In some embodiments at least the activator is dispensed according to the above protocol for the entire method until the array of biopolymers is prepared. In some embodiments at least the nucleotide monomers and activator are dispensed for a portion of the method in accordance with the above protocol until the array of biopolymers is patterned and the preparation of the array is completed by any suitable protocol.

In some embodiments the invention is directed to an apparatus for preparing an array of biopolymer features on a surface of a substrate. The apparatus comprises (a) at least one drop dispensing device comprising one or more drop dispensers each comprising multiple nozzles, (b) a mechanism for moving the drop dispensing device or a substrate relative to one another in one or more drop dispensing cycles to dispense biopolymer forming reagents to the surface of the substrate, and (c) a computer comprising a computer program to control the movement of the mechanism and the activation of the nozzles of the drop dispensing device in a protocol such that, for at least one of the biopolymer forming reagents and for at least a portion of the method, drops of reagents are dispensed to feature locations from at least two different nozzles for each reagent in at least two different passes of a drop dispensing device.

In some embodiments the computer program controls the activation of the nozzles where the two different nozzles are on the same drop dispenser. In some embodiments the apparatus comprises two different drop dispensers for each reagent dispensed and the computer program controls the activation of the nozzles where the two different nozzles are on the two different drop dispensers.

In some embodiments of an apparatus, the biopolymer features are polynucleotide features and the biopolymer forming reagents comprises nucleotide monomers and activators. In some embodiments of the above apparatus, the computer program controls the activation of the orifices to dispense at least the nucleotide monomers and activator according to the above protocol for the entire method until the array of biopolymers is prepared. In some embodiments of the above apparatus, the computer program controls the activation of the orifices to dispense at least the activator according to the above protocol for the entire method until the array of biopolymers is prepared. In some embodiments of the above apparatus, the computer program controls the activation of the orifices to dispense at least the nucleotide monomers and activator for a portion of the method in accordance with the above protocol until the array of biopolymers is patterned and the preparation of the array is completed by any suitable protocol.

### BRIEF DESCRIPTION OF THE DRAWINGS

The following figures are included to better illustrate the embodiments of the apparatus and techniques of the present invention. The figures are not to scale and some features may be exaggerated for the purpose of illustrating certain aspects or embodiments of the present invention.

FIG. 1 is a perspective view of a substrate bearing multiple arrays.

FIG. 2 is an enlarged view of a portion of FIG. 1 showing some of the identifiable individual regions (or “features”) of a single array of FIG. 1.

FIG. 3 is an enlarged cross-section of a portion of FIG. 2.

FIG. 4 is a schematic drawing depicting an embodiment in accordance with the present methods.

FIG. 5 is a schematic drawing depicting another embodiment in accordance with the present methods.

FIG. 6 is a schematic drawing depicting another embodiment in accordance with the present methods.

FIG. 7 is a schematic drawing depicting an apparatus in accordance with the present disclosure.

### DESCRIPTION OF SPECIFIC EMBODIMENTS

The present embodiments may be employed to reduce the number of errors in the preparation of an array of biopolymers. Such errors may be, for example, those resulting from failure of one or more nozzles to deliver reagents to the desired locations. The nozzle may be clogged, malfunctioning, and the like.

Various protocols may be performed to achieve a reduction in number of errors. The present protocols may be carried out without the need for detecting errors during an array fabrication so that the error may be corrected by redepositing reagent at the error site. The present protocols may be carried out for all of the monomer reagents and/or activating agents employed in the synthesis or the protocols may be carried out for one or more of the monomer reagents and/or the activating agents. The protocol may be conducted for the entire method for the preparation of an array of biopolymers.

On the other hand, the protocol may be carried out for only a portion of the method for the preparation of an array of biopolymers. In this instance the cycles employing the dispensing protocol described herein are repeated a sufficient number of times until the array of biopolymers is patterned, i.e., to prepare the array of biopolymer features having a desired level of hydrophilicity in the feature areas for which the above protocol is carried out. This is discussed in more detail below. When the protocol is followed for less than all of the monomer reagents and/or activating agents, the remaining reagents may be dispensed to the surface of the substrate by known protocols. When the protocol is followed for less than the entire method for preparation of an array of biopolymers, the remainder of the preparation may be carried out by known protocols. The present embodiments will be discussed in more detail below.

In some embodiments, a protocol is employed wherein in each cycle drops of reagents are dispensed to feature locations from at least two different nozzles for each reagent in at least two different passes of a drop dispensing device. The at least two different nozzles may be present on the same drop dispenser or they may be present on two different drop dispensers. These embodiments will be discussed in more detail below. In some embodiments a combination of the above approaches may be employed. For example, in some of the cycles of the method the reagents are dispensed according to a protocol involving two different nozzles on the same drop dispenser and for some of the cycles of the method the reagents are dispensed from two different drop dispensers. It is also contemplated to employ both of the above protocols in the same cycle.

The following protocols are examples, by way of illustration and not limitation, for protocols in accordance with the present methods. In some embodiments every monomer reagent and/or activating reagent has a redundant dispenser at all times for all of the locations to which these reagents are dispensed. In some embodiments, one dispenser is used per reagent and each dispenser has more than one nozzle. In some embodiments only one or more activating agents have redundant dispensers at all times for all locations. In some embodiments redundant dispensers are used only during part of the method, usually the beginning, to impose a feature size by the process of array patterning. Once the surface is patterned, redundant dispensers are discontinued and large feature sizes are obtained with smaller volumes of reagents applied according to known protocols.

Before the subject invention is described further, it is to be understood that the invention is not limited to the particular embodiments of the invention described herein, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs. Any recited method can be carried out in the order of events recited or in any other order that is logically possible. Reference to a singular item, includes the possibility that there are plural of the item present. “May” refers to optionally.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

In some embodiments, the present invention provides methods for preparing substrates having an array of features bound to at least one surface of the substrate. The features generally comprise chemical compounds, usually, polymeric chemical compounds, for example, biopolymers, formed from polymer subunits, for example, nucleotide reagents or amino acid reagents.

For example, various ways may be employed to introduce reagents for producing an array of polynucleotides on the surface of a substrate such as a glass substrate. Such methods are known in the art. Many embodiments of such methods employ droplet dispensing devices. The phrase “droplet dispensing device” includes any device that dispenses drops of fluid, usually, a liquid. The droplet dispensing device normally includes a reagent source or manifold or reservoir as well as reagent lines that connect the source to fluid dispensing nozzles of a drop dispenser and the like. The “reservoir” may be any container that is suitable for containing a fluid reagent. In many embodiments, the droplet dispensing devices comprise one or more drop dispensers each comprising a plurality of nozzles. In some embodiments the nozzles are aligned in at least one row. Alternatively, the nozzles may be aligned in at least two rows, at least three rows, at least four rows, and so forth. Usually, the maximum number of rows is about 14. Preferably, the number of rows of nozzles is about 4 to about 8.

The phrase “pulse jet” refers to a device that can dispense drops by delivering a pulse of pressure (such as by a piezoelectric or thermoelectric element) to liquid adjacent an outlet or orifice such that a drop will be dispensed therefrom. As is well known in the art, the amount of fluid that is expelled in a single activation event of a pulse jet, can be controlled by changing one or more of a number of parameters, including the orifice diameter, the orifice length (thickness of the orifice member at the orifice), the size of the deposition chamber, and the size of the heating element, and so forth. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s (meter/second), usually more than about 10 m/s, and may be as great as about 20 m/s or greater. Droplet dispensing devices include, for example, pulse jets, and so forth. “Fluid” is used herein primarily to reference a liquid.

In one specific embodiment a droplet dispensing device comprises one or more drop dispensers sometimes referred to as dispensing heads. Each dispensing head or drop dispenser carries hundreds of ejectors or nozzles to deposit droplets of a reagent. Each ejector may be in the form of an electrical resistor operating as a heating element under control of a processor (although piezoelectric elements could be used instead). Each orifice with its associated ejector and a reservoir chamber, acts as a corresponding pulse-jet with the orifice acting as a nozzle. In this manner, application of a single electric pulse to an ejector causes a droplet to be dispensed from a corresponding orifice (or larger droplets could be deposited by using multiple pulses to deposit a series of smaller droplets at a given location).

The drop dispenser or dispensing head may be of a type commonly used in an ink jet type of printer and may, for example, have one hundred fifty drop dispensing orifices in each of two parallel rows, six chambers for holding solutions of nucleotide precursors communicating with the three hundred orifices, and three hundred ejectors which are positioned in the chambers opposite a corresponding orifice. Thus, there are three hundred pulse jets in this exemplary configuration, although it will be appreciated that a dispensing head could, for example, have more or less pulse jets as desired (for example, at least ten or at least one hundred pulse jets or more). In this manner, application of a single electric pulse to an ejector causes a droplet to be dispensed from a corresponding orifice. Certain elements of the dispensing head can be adapted from parts of a commercially available thermal inkjet print head device available from Hewlett-Packard Co. as part no. HP51645A. The foregoing dispensing head and other suitable dispensing head designs are described in more detail in U.S. Pat. No. 6,461,812 entitled “A Multiple Reservoir Ink Jet Device for the Fabrication of Biomolecular Arrays,” the relevant disclosure of which is incorporated herein by reference.

As mentioned above, the droplet dispensing devices comprise one or more drop dispensers each comprising a plurality of nozzles for dispensing biopolymer subunit precursors to a surface of a substrate on which the array of features is to be synthesized. Each nozzle of the droplet dispenser is normally in fluid communication with a source of a polymer forming unit or polymer forming reagent. For each drop dispenser there is spacing between the nozzles, which is sometimes referred to as the “native spacing.” This spacing is dependent on the nature of the drop dispensing device. In some embodiments the native spacing may be about 50 microns, about 60 microns, about 75 microns, about 100 microns, about 212 microns, about 254 microns, about 508 microns, about 600 microns and so forth.

In a method in accordance with embodiments of the present invention, a reagent for forming the chemical compounds, such as, for example, biopolymer forming reagent, e.g., a nucleotide reagent or a polynucleotide reagent, is deposited on the surface of the support as discussed above. Usually, a deposition sequence is initiated to deposit the desired fluid droplets containing the reagents on the surface of a support to provide dried drops on the surface according to the predetermined arrangement of the target, each with respective target locations and dimensions. In this sequence a processor causes a positioning system to position a head facing the surface of the support at an appropriate distance from the surface. The processor then causes the positioning system to scan the drop dispensing device comprising one or more drop dispensers across the surface line by line (or in some other desired pattern), while coordinating activation of the ejectors in the drop dispenser so as to dispense droplets in accordance with the target pattern. The processor can repeat the load and dispensing sequences one or more times until the drop dispenser has dispensed the desired number of droplets in accordance with the particular reaction step of the array formation.

Generally, in drop deposition techniques a pass or swath of a drop dispensing device is employed for each cycle of drop deposition. The term “swath” means the pass of a drop dispensing device across at least a portion of a substrate to deposit drops of a reagent at predetermined locations on the surface of the substrate.

“Trigger point” means the point in time at which all or a portion of the nozzles of a drop dispensing device are activated to dispense a drop of reagent.

During the drop dispensing process, e.g., printing process, the movement of the drop dispensing device such as, e.g., print stages, generates quadrature pulses. The term “quadrature pulse” means an electrical signal generated by a moving mechanism where each pulse is related to the distance moved. Quadrature pulses may be spaced from about 100 nm to about 10 mm apart.

The quadrature pulses are counted by a control board such as, for example, a printhead control board, as they occur. When the quadrature pulse count reaches the number specified for a particular Trigger Point setting, the control board that controls the dispensing of the drop dispensing device tells the drop dispensing device which nozzles to fire and which to inhibit and, then, stimulates the crystals in the drop dispensing device with the specified waveform causing fluid to be ejected at the appropriate location.

The phrase “control board” means an electronic assembly that can be programmed with a list of trigger points occurring at specific quadrature pulses, activating particular sets of nozzles and using particular waveforms to cause fluid to be ejected from the nozzles.

Features of the array may be arranged, for example, in rectilinear rows and columns. This is particularly attractive for single arrays on a substrate. However, other configurations are possible as discussed in more detail below. The following discussion is directed primarily to features of an array that are arranged in rows and columns by way of illustration and not limitation. In the description herein the terms “x-axis,” “y-axis” and “z-axis” or “X-axis,” “Y-axis” and “Z-axis” reference distinct axes and, preferably, a coordinate system that is orthogonal, i.e., a Cartesian coordinate system. This coordinate system relates to the direction of travel of a drop dispensing device and a substrate relative to one another in the preparation of an array of features arranged in columns and rows.

The phrase “drop dispensing relationship” as used above refers to bringing the drop dispensing device and the substrate into proximity such that drops of fluid may be dispensed to the surface of the substrate at one or more locations on the surface whether or not drops of fluid are actually dispensed to the surface of the substrate. For example, an apparatus and accompanying hardware (for example, a computer) and software (for example, a computer program product) may be programmed such that the drop dispensing device and the substrate are brought into drop dispensing relationship in a protocol such as that discussed above.

The drop dispensing device dispenses polymer-forming reagents to different substrates sequentially until all polymer subunits are deposited for a drop-dispensing step of a cycle of the synthesis, which is sometimes referred to herein as a “particular cycle.” The term “particular cycle” refers to a cycle comprising the steps of dispensing drops of polymer forming reagents to predetermined locations on the surface of the substrate, dispensing an activating agent to the predetermined feature locations, and subjecting the predetermined locations to reagents for preparing the locations for a next drop dispensing step. A drop dispensing step involves dispensing drops of a polymer forming reagent to predetermined locations on the surface of the substrate followed by dispensing drops of a different polymer forming reagent to other predetermined locations and so forth until all polymer forming reagents are dispensed for the dispensing step of a cycle of the synthesis.

As discussed herein by way of example, desired polymer subunits, such a monomers, are dispensed to extend a growing polymer chain of each polymer to be synthesized at a specific location or feature area on the surface of a substrate by one polymer subunit. For example, consider a cycle in the synthesis of oligonucleotides on the surface of a substrate. The growing chain for each oligonucleotide is 8 nucleotides in length. In a particular cycle, various nucleotide subunits are dispensed dropwise to predetermined locations on the surface of the substrate to extend the growing chain to 9 nucleotides in length. Accordingly, drops of one of the nucleotide reagents are dispensed to predetermined locations to add the ninth nucleotide at these locations and then drops of another nucleotide reagent are dispensed to other predetermined locations to add the ninth nucleotide at these other locations. The cycle of the synthesis is not complete until all locations have a ninth nucleotide. An activating agent is also dispensed to the predetermined locations as part of each cycle. Each nucleotide addition to all of the locations is sometimes referred to in the art as forming a layer. Thus, in each completed cycle a layer of nucleotides is formed on the surface of the substrate. In the example above, the cycle produced the ninth layer.

By “sequentially” is meant that each drop dispensing relationship among respective substrate surfaces follows the establishment of a previous drop dispensing relationship until all drop dispensing relationships between respective substrate surfaces have been established to deposit the reagents necessary for a particular cycle of the synthesis.

The drop dispensing device may be employed with other drop dispensing devices in the form of drop dispensing modules. The modules are generally a housing or structural element to which the drop dispensers are attached and may comprise components for providing liquid communication between the drop dispensers and a source of reagents, which may or may not be part of the module. The drop-dispensing module is a housing structure designed to hold/secure a drop dispenser or an assembly thereof. The housing is therefore configured to engagingly fit with or connect to a drop dispenser or an assembly thereof. In principle, the housing is configured to fit with any type of drop dispenser assembly, including pulse jet assemblies, such as piezoelectric and thermal pulse jet assemblies.

The modules may also include reagent sources or manifolds as well as reagent lines that connect the source to fluid dispensing nozzles and the like. The modules may also comprise one or more pumps for moving fluid and may also comprise a valve assembly and a manifold. The fluids may be dispensed by any known technique. Any standard pumping technique for pumping fluids may be employed in the dispensing device. For example, pumping may be by means of a peristaltic pump, a pressurized fluid bed, a positive displacement pump, e.g., a syringe pump, and the like.

The phrase “polymer forming reagents” includes polymer subunits as well as other reagents necessary for adding a polymer subunit to a growing polymer chain on the surface of a substrate. Such other reagents include, for example, activator reagents, and the like. As may be appreciated, the nature of the other reagents depends on the nature of the polymers formed, the polymer forming reagents, and so forth.

A “polymer subunit” is a chemical entity that can be covalently linked to one or more other such entities to form an oligomer or polymer. The polymer subunit may be a monomer or a chain of monomers. Examples of monomers include nucleotides, amino acids, saccharides, peptoids, and the like and chains comprising nucleotides, amino acids, saccharides, peptoids and the like. The chains may comprise all of the same component such as, for example, all of the same nucleotide or amino acid, or the chain may comprise different components such as, for example, different nucleotides or different amino acids. The chains may comprise at least 2, at least 3, at least 4, at least 5, at least 6, at least 7, at least 8, at least 9, at least 10, and so forth, monomer units and may be in the range of about 2 to about 2000, or about 2 to about 200, or about 2 to about 100 monomer units. In general, the polymer subunits, for example, may have first and second sites (e.g., C-termini and N-termini, or 5′ and 3′ sites) suitable for binding of other like monomers by means of standard chemical reactions (e.g., condensation, nucleophilic displacement of a leaving group, or the like), and a diverse element that distinguishes a particular monomer from a different monomer of the same type (e.g., an amino acid side chain, a nucleotide base, etc.). The initial substrate-bound monomer is generally used as a building block in a multi-step synthesis procedure to form a complete polymer, such as in the synthesis of oligonucleotides, polynucleotides, oligopeptides, polypeptides, oligosaccharides, polysaccharides, and the like.

As mentioned above, in some embodiments of the present invention, a nozzle firing protocol is employed for one or more biopolymer forming units and/or one or more activating agents. The protocol may be carried out for the particular reagents for all of the cycles of the method, i.e., for the entire method, until the array of biopolymers of desired length is prepared. Alternatively, the protocol may be carried out for less than all, or a portion, of the cycles, i.e., a portion of the method, until the array of biopolymers is patterned and the remainder of the cycles may be conducted according to known protocols until the array of biopolymers of desired length is prepared. In embodiments where less than all of the biopolymer forming units and/or less than all activating agents employed in the synthesis are dispensed according to the present protocols, known dispensing protocols are employed for those biopolymer forming units and/or activating agents not dispensed according to the present protocols. Furthermore, where the present protocols are followed for only a portion of the total number of cycles, known dispensing protocols are employed for the remaining cycles.

The phrase “array of biopolymers is patterned” may be explained as follows. The synthesis of the biopolymer array on the surface of the solid support substantially modifies the physical properties of that solid support where the biopolymer is physically attached to the solid support. As the length of the biopolymers increases at biopolymer feature locations on the surface, the physical properties of the biopolymer features become increasingly dissimilar from the physical properties of the inter-feature areas of the surface. In particular, the surface energy of the biopolymer feature areas increases and the features become more hydrophilic relative to the inter-feature area, hence creating a patterned surface having areas of unchanged surface energy and areas of increased surface energy. Thus, the hydrophilicity at the biopolymer feature locations increases with increase in length of the biopolymer features.

The method in accordance with the present disclosure is employed at least until a desired level of hydrophilicity or biopolymer length is obtained. The length of the biopolymer features, i.e., the number of monomer units such as nucleotides that achieves the desired level of hydrophilicity, is dependent on the nature of the biopolymer, the nature of the biopolymer forming reagents, and the like. The number may be about 1 to about 60, or about 2 to about 50, about 2 to about 40, or about 3 to 5 about 30, or about 3 to about 20, or about 4 to about 20, or about 3 to about 10, or about 4 to about 10, or about 5 to about 30, or about 5 to about 20, or about 5 to about 15, or about 5 to about 10, and so forth.

Once a desired level of hydrophilicity is obtained, single drop deposition of biopolymer reagent may then be utilized and the drop that is deposited at each predetermined feature location spreads to cover substantially all of the feature area for a biopolymer feature in question because a requisite degree of hydrophilicity is achieved. In an example by way of illustration and not limitation, a biopolymer feature may be formed by depositing multiple drops in accordance with the present method. For instance, deposition of two drops of biopolymer forming reagent or monomer unit is employed to form a 65-micron feature until the length of the biopolymer feature is 15 monomer units. Then, a single drop of biopolymer reagent may be deposited at the feature location and the drop spreads to cover substantially all of the 65 microns.

Accordingly, the method in accordance with the invention may be practiced by using a protocol for at least one of the biopolymer forming reagents and for at least a portion of the method wherein drops of one or more biopolymer forming reagents are dispensed to a surface of a substrate in multiple cycles to synthesize the biopolymers. In the present dispensing protocol, each different biopolymer forming reagent is dispensed from a separate drop dispenser of a drop dispensing device and each drop dispenser comprises multiple nozzles. Multiple passes of the drop dispensing devices are employed for each cycle. In each cycle drops of reagents are dispensed to feature locations from at least two different nozzles for each reagent in at least two different passes of a drop dispensing device. This protocol is followed until the feature areas at which the biopolymer forming reagents are deposited reach a desired level of hydrophilicity, i.e., a level of hydrophilicity wherein subsequent single drop deposition to the feature area results in spreading the of drop to cover substantially all of the feature area. “Substantially all of the feature area” means that at least 90%, at least 95%, at least 97%, at least 98%, at least 99%, or 100% of the feature area is covered by a single drop of biopolymer forming reagent.

The phrase “known” protocol refers to the deposition of a single drop of biopolymer forming reagent to a feature location. Generally, the volume of this single drop of reagent is less than the volume of the drop that would otherwise be necessary to cover a given or predetermined feature area. In this way, reagents are conserved when compared to utilizing single drop deposition for the entire method for preparing an array of biopolymer features of a predetermined or desired length because the drop volume would be larger. The drop volume for this single drop approach, when the single drop approach follows the deposition of features using a protocol in accordance with the present methods is less than about 95%, about 90%, less than about 85%, less than about 80%, less than about 70%, less than about 60%, less than about 55%, less than about 50%, of the volume of the drop when the present protocols are not employed to pattern the array surface. The volume of the drop in the above approach is about 40% to about 95%, about 50% to about 90%, about 50% to about 80%, about 50% to about 70% of the volume of a single drop when the present protocols are not employed to pattern the array surface.

The phrase “less than all the cycles” is defined in relation to, and is dependent on, the patterning of the array as discussed above. The number of cycles for patterning the array may be about 1 to about 200, or about 1 to about 60, about 2 to about 20, or about 3 to about 15, or about 3 to about 10, or about 3 to about 5, and the like.

In each cycle of the present protocols, drops of reagents are dispensed to feature locations from at least two different nozzles for each reagent in at least two different passes or swaths of a drop dispensing device. While the discussion herein is directed primarily to the delivery of drops of reagent from two different nozzles, the number of different nozzles for each feature location may be 3, or 4, or 5, or 6, or 7, or 8, or 9, or 10 or more. The maximum number of different nozzles for each feature location is dependent on a number of factors such as, for example, the size of the drop dispenser, the number of feature locations serviced by the drop dispenser, the pattern of nozzle firing, the number of nozzles, the number of drop dispensers that can fit in the manufacturing unit where printing is performed, the throughput required for the printing process, and so forth.

The phrase “swath number” means the number of swaths of a drop dispensing device in a particular drop deposition cycle to deposit drops of reagents at feature locations on a substrate. The swath number is usually an integer and in some embodiments may be 1, or 2, or 3, or 4, or 5, or 6, or 7, or 8, or 9, or 10, and so forth.

### Protocol Involving Two Different Nozzles of the Same Drop Dispenser

In some embodiments the at least two different nozzles that dispense to the same feature location are present on the same drop dispenser of the drop dispensing device. These embodiments may be realized in a number of different ways.

For example, in some embodiments on the first pass or swath of the drop dispensing device, the drop dispenser is in a designated spatial relationship such as, for example, alignment, position, acceleration and deceleration ramps, print direction, velocity, or the like, with respect to the rows and columns of features on the substrate surface. All of the nozzles of a drop dispenser that are to be triggered for the synthesis cycle in question are triggered to deliver drops of a reagent to the surface of a substrate at specified feature locations. The nozzles that are triggered are determined by the specified feature locations that are to receive a drop of reagent as well as the chosen protocol and the pattern of nozzle firing (“nozzle firing pattern”) is determined by the pattern of the specified feature locations. Accordingly, a nozzle firing pattern is employed in the first pass to deliver a drop of reagent to the desired feature locations. In known approaches, it is typical to employ the same firing pattern for all of the swaths of the particular synthesis cycle being conducted.

In accordance with embodiments of the present invention, in a second pass of the drop dispensing device for the cycle in question, the drop dispenser is moved to change its spatial relationship with respect to the rows and columns of features. For example, the change in spatial relationship may be along an x-axis or a y-axis or a combination thereof. The change in spatial relationship may be viewed in relation to the rows or columns of the nozzles of the drop dispenser. For example, the spatial relationship of the drop dispenser may be changed by moving the drop dispenser so that a different row of nozzles is aligned with a row of feature locations that is different that the row of feature locations of the first pass.

In the second pass, an adjusted nozzle firing pattern is implemented so that a different nozzle delivers a drop of reagent to a feature location to which a drop of reagent was delivered in the first swath. An adjusted nozzle firing pattern is a nozzle firing pattern wherein the pattern of nozzles that are triggered for a dispenser differs from the pattern of nozzles triggered for another nozzle firing pattern to deliver reagent drops to specified feature locations that are the same for delivery of the reagent in question during a particular cycle of a synthesis of biopolymers. The nature of the adjusted nozzle firing pattern is determined by several factors such as, for example, the feature locations that were to receive reagent in the first swath, the alteration in the spatial relationship of the drop dispensing device to the feature locations on the surface of the substrate, and so forth. The change in the nozzle firing pattern is implemented by appropriate software that enables a computer to communicate instructions to the drop dispensing device.

After the adjustment in spatial relationship of the drop dispenser, a second pass of the drop dispensing device is carried out for the same cycle of the synthesis in which all of the nozzles of a drop dispenser to be triggered are triggered according to a nozzle firing pattern for the second pass to deliver drops of the same reagent to the surface of the substrate at specified feature locations so that a different nozzle delivers reagent to a feature location to which reagent was to be delivered to in the first pass. As a result, if one or more nozzles failed to deliver reagents to one or more feature locations in the first pass, the change in spatial relationship of the dispensing device with respect to the surface of the substrate in the second pass as well as the change in the nozzle firing pattern results in delivery of reagent to any feature locations that did not receive reagent on the first pass. This occurs because any nozzle that failed to deliver reagent is moved to a position such that the feature location receiving the reagent has already received a drop of reagent during the first pass. Likewise, a feature location that did not receive a drop of reagent during the first pass receives a drop of the reagent from a different, functioning nozzle on the second pass.

After the adjustment in orientation of the drop dispenser, and depending on the swath number for the particular cycle of the synthesis in question, a third pass of the drop dispensing device may or may not be carried out for the same cycle of the synthesis. If a third pass is employed, all of the nozzles of a drop dispenser to be triggered are triggered according to an adjusted nozzle firing pattern to deliver drops of the same reagent to the surface of the substrate at specified feature locations. The nozzle firing pattern may be the same as, or different from, that of the first pass and/or the second pass. However, in accordance with the present embodiments, the feature locations for each swath are those that are to receive reagent for the particular synthesis cycle. The passes may be continued in a similar manner until the swath number is achieved for a particular cycle of the synthesis. Changing the spatial relationship of the drop dispensing device to the feature locations on the surface of the substrate, as well as the corresponding nozzle firing pattern, for one, two, three, etc., up to all of the swaths of the cycle of the synthesis enhances the ability to avoid errors in the delivery of reagent to any one feature location.

In one illustrative example based on the above method, on the first pass or swath of the drop dispensing device, the drop dispenser is in a designated alignment with respect to the rows and columns of features on the substrate surface. All of the nozzles of a drop dispenser to be triggered are triggered according to a nozzle firing pattern to deliver drops of a reagent to the surface of a substrate at specified feature locations. Then, the drop dispensing device is moved, for example, in the X direction only, so that the alignment of the nozzles with respect to a column of feature locations differs from the alignment of the nozzles for the first pass. Again, all of the nozzles of a drop dispenser to be triggered are triggered according to an adjusted nozzle firing pattern to deliver drops of a reagent to the surface of a substrate at specified feature locations. However, because of the change in the alignment of the drop dispensing device and the nozzle firing pattern, a different nozzle delivers the same reagent to a specified feature location that was to receive reagent in the first swath. The drop dispensing device may be moved again, for example, in the X direction only, so that the alignment of the nozzles with respect to a column of feature locations differs from the alignment for the first pass and/or the second pass of the drop dispensing device. An appropriate nozzle firing pattern is implemented as discussed above to deliver reagents to specified feature locations on the surface of the substrate.

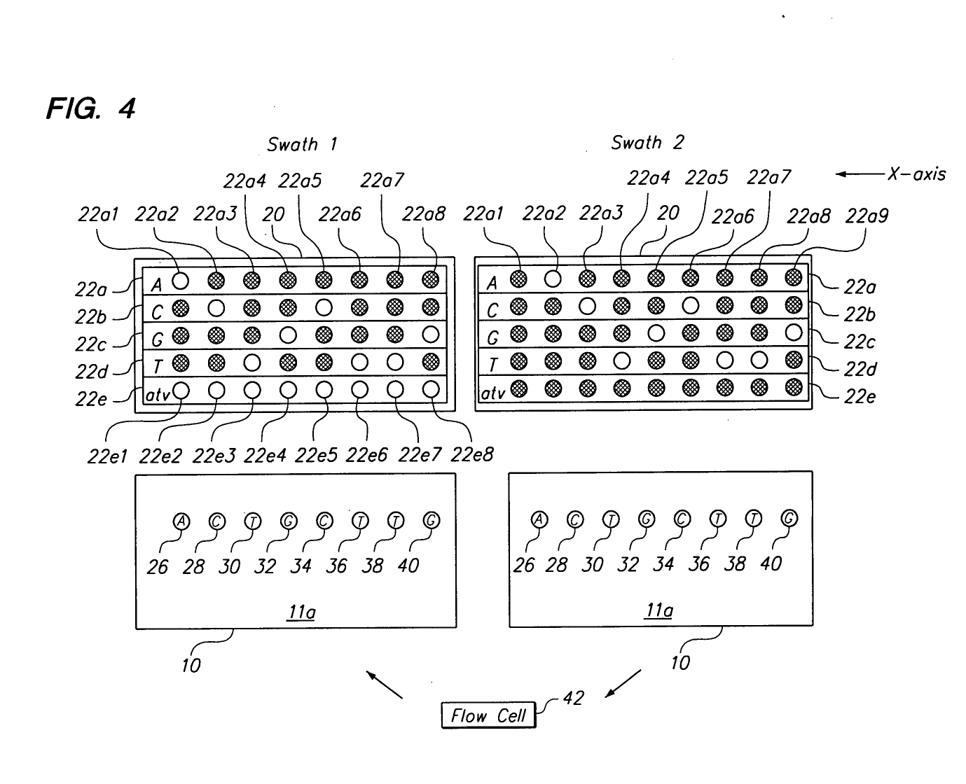

An illustrative example in accordance with the above embodiments is illustrated in FIG. 4 wherein two passes (Swath 1 and Swath 2) of a drop dispensing device 20 are depicted for the drop deposition step of a synthesis cycle. Drop dispensing device 20 is moved into a drop dispensing relationship with surface 11a of substrate 10. Drop dispensing device 20 is shown, for purposes of illustration and not limitation, with five drop dispensers 22a, 22b, 22c, 22d and 22e. Each drop dispenser dispenses a different nucleotide monomer reagent or an activator (atv), which may be, for example, tetrazole. The nucleotide monomer reagents are in the form of phosphoramidate reagents for synthesizing an array of polynucleotides on the surface of a substrate. Thus, as depicted, drop dispenser 22a dispenses nucleotide monomer dA, drop dispenser 22b dispenses nucleotide monomer dC, drop dispenser 22c dispenses nucleotide monomer dG, drop dispenser 22d dispenses nucleotide monomer dT and drop dispenser 22e dispenses activator. Each of the dispensers comprises multiple orifices or nozzles, most of which are not shown. In FIG. 4, only a few of the nozzles for each of the drop dispensers are shown for purposes of convenience. The nozzles are designated by consecutive numbers, which follow the identifying designation for each of the drop dispensers. For example, the nozzles for drop dispenser 22a are designated 22a1, 22a2, 22a3, 22a4, 22a5, 22a6, 22a7, 22a8, 22a9 and so forth.

On the first pass or Swath 1 of drop dispensing device 20 over substrate 10, each of the drop dispensers is in a designated alignment with respect to the rows and columns of features on substrate surface 11a. In FIG. 4, only a single row of features is shown for purposes of convenience. It is to be understood that an array normally has many more features as discussed herein below, which may be arranged, for example, in various rows and columns. The feature locations depicted are designated 26, 28, 30, 32, 34, 36, 38 and 40, respectively. First, drops of activator reagent are dispensed to predetermined locations on surface 11a of substrate 10 followed by dispensing of particular polymer forming reagents dA, dC, dG, dT, respectively. Accordingly, all of the nozzles of a drop dispenser to be triggered are triggered according to a nozzle firing pattern to deliver drops of a particular reagent to the surface of a substrate at specified feature locations. In the example shown, the nozzle firing pattern is as follows: nozzle 22a1 to feature location 26, nozzle 22b2 to feature location 28, nozzle 22d3 to feature location 30, nozzle 22c4 to feature location 32, nozzle 22b5 to feature location 34, nozzle 22d6 to feature location 36, nozzle 22d7 to feature location 38, nozzle 22c8 to feature location 40 in Swath 1 of drop dispensing device 20.

Then, the drop dispensing device is moved, for example, in the X direction only, so that the alignment of the nozzles with respect to a column of feature locations differs from the alignment of the nozzles for the first pass. In Swath 2, activator is not dispensed. Rather, another drop of nucleotide monomer reagent is delivered to a designated feature location. Accordingly, all of the nozzles of a drop dispenser to be triggered are triggered according to an adjusted nozzle firing pattern, i.e., the nozzle firing pattern for Swath 2, to deliver drops of a reagent to the surface of a substrate at specified feature locations. However, because of the change in the alignment of the drop dispensing device and the nozzle firing pattern, a different nozzle delivers the same reagent to a specified feature location that was to receive reagent in Swath 1. In the example shown, the nozzle firing pattern is as follows: nozzle 22a2 to feature location 26, nozzle 22b3 to feature location 28, nozzle 22d4 to feature location 30, nozzle 22c5 to feature location 32, nozzle 22b6 to feature location 34, nozzle 22d7 to feature location 36, nozzle 22d8 to feature location 38, nozzle 22c9 to feature location 40 in Swath 2 of drop dispensing device 20.

Although not shown in the figure, the drop dispensing device may be moved again, for example, in the X direction only, so that the alignment of the nozzles with respect to a column of feature locations differs from the alignment for the first pass and/or the second pass of the drop dispensing device. An appropriate nozzle firing pattern is implemented as discussed above to deliver reagents to specified feature locations on the surface of the substrate.

Following the dispensing of reagents for the dispensing step of the cycle in question, substrate 10 is placed in flow cell 42 where it is subjected to various treatment steps of the synthesis cycle such as, for example, blocking or capping, oxidation and deblocking or detritylation as mentioned above. In this embodiment the treatment is accomplished by treating the entire substrate (“flooding”) with a liquid layer of the appropriate reagent. The above protocol is repeated in a next cycle to deposit the appropriate polymer forming reagents for the next cycle in the synthesis. The cycles are repeated a sufficient number of times (cycle number) until the desired array of biopolymer features has been synthesized or until the array has been patterned. Final deprotection of nucleoside bases can be accomplished thereby producing the final array product. The cycle number is dependent on the length of the biopolymer features, the nature of the biopolymer, the desired application, sensitivity and specificity of the array device, and the like.

### Protocol Involving Two Different Nozzles on Two Different Drop Dispensers

In some embodiments the at least two different nozzles that dispense to the same feature location are present on two different drop dispensers of the drop dispensing device. These embodiments may be realized in a number of different ways. In some embodiments there are two different drop dispensers for each of the reagents to be dispensed in a dropwise manner.

As an example of the above approach, in some embodiments on the first pass or swath of the drop dispensing device, one drop dispenser (arbitrarily designated as a first drop dispenser) is in a designated spatial relationship with respect to the rows and columns of features on the substrate surface. All of the nozzles of a drop dispenser that are to be triggered for the synthesis cycle in question are triggered to deliver drops of a reagent to the surface of a substrate at specified feature locations. The nozzles that are triggered are determined by the specified feature locations that are to receive a drop of reagent as well as the chosen protocol. Accordingly, a nozzle firing pattern is employed in the first pass to deliver a drop of reagent to each of the desired feature locations.

In accordance with embodiments of the present invention, another drop dispenser (arbitrarily designated as a second drop dispenser) is present on the same dispensing device. During the first pass or swath of the second drop dispenser is in a designated spatial relationship with respect to the rows and columns of features on the substrate surface so that the second dispenser can dispense the same reagent, as that dispensed by the first dispenser, from selected nozzles to the same locations as those to which the first dispenser dispensed. All of the nozzles of the second drop dispenser that are to be triggered for the synthesis cycle in question are triggered to deliver drops of a reagent from the second dispenser to the surface of a substrate at specified feature locations. The nozzles that are triggered are determined by the specified feature locations that are to receive a drop of reagent as well as the chosen protocol. Accordingly, a nozzle firing pattern is employed for the second dispenser in the first pass to deliver a drop of reagent to the desired feature locations. The control of the nozzle firing pattern for the first and the second dispensers is implemented by appropriate software that enables a computer to communicate instructions to the drop dispensing device.

The use of the phrases “first dispenser,” “second dispenser” and the like is not meant to imply a particular order of the dispensers on the drop dispensing device or any other order. The designation relates to the order of firing of the particular dispensers involved. Furthermore, there may be additional redundant dispensers for each reagent on the drop dispensing device depending on the size tolerances of the drop dispensing device, the size tolerance of the manufacturing unit where drop dispensing is performed, the size of the array features, and the like.

In other embodiments in accordance with the above dispensing protocol, the at least two different dispensers may be present on different dispensing devices, which are under appropriate computer control to achieve the desired dispensing protocol. For example, one drop dispensing device may comprise five dispensers, one each for five different reagents and another drop dispensing device may also comprise five dispensers similar to those of the first drop dispensing device. Depending on which reagent is to be delivered to specified locations on a surface of a substrate in a particular cycle of the synthesis, the selected reagent is delivered in accordance with the present protocol from a dispenser on the first drop dispensing device and a dispenser on the second drop dispensing device.

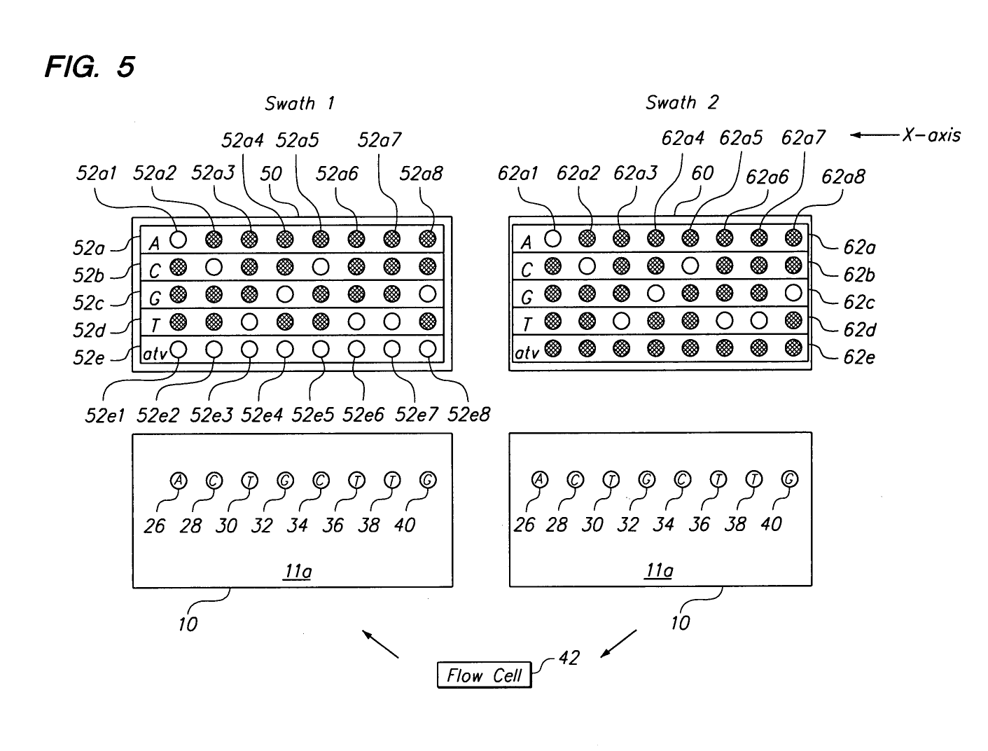

An illustrative example in accordance with the above embodiments is illustrated in FIG. 5 wherein two passes (Swath 1 and Swath 2) are carried out using two different drop dispensing devices 50 and 60 and are depicted for the drop deposition step of a synthesis cycle. Drop dispensing device 20 is moved into a drop dispensing relationship with surface 11a of substrate 10. Drop dispensing device 50 is shown for purposes of illustration and not limitation with five drop dispensers 52a, 52b, 52c, 52d and 52e. Drop dispensing device 60 is shown for purposes of illustration and not limitation with five drop dispensers 62a, 62b, 62c, 62d and 62e. Each drop dispenser dispenses a different nucleotide monomer reagent or an activator (atv), which may be, for example, tetrazole. The nucleotide monomer reagents are in the form of phosphoramidate reagents for synthesizing an array of polynucleotides on the surface of a substrate. Thus, as depicted, drop dispenser 52a dispenses nucleotide monomer dA, drop dispenser 52b dispenses nucleotide monomer dC, drop dispenser 52c dispenses nucleotide monomer dG, drop dispenser 52d dispenses nucleotide monomer dT and drop dispenser 52e dispenses activator and drop dispenser 62a dispenses nucleotide monomer dA, drop dispenser 62b dispenses nucleotide monomer dC, drop dispenser 62c dispenses nucleotide monomer dG, drop dispenser 62d dispenses nucleotide monomer dT and drop dispenser 62e dispenses activator. Each of the dispensers comprises multiple orifices or nozzles, most of which are not shown. In FIG. 5, only a few of the nozzles for each of the drop dispensers are shown for purposes of convenience. The nozzles are designated by consecutive numbers, which follow the identifying designation for each of the drop dispensers. For example, the nozzles for drop dispenser 52a are designated 52a1, 52a2, 52a3, 52a4, 52a5, 52a6, 52a7, 52a8 and so forth.

On the first pass or Swath 1 of drop dispensing device 50 over substrate 10, each of the drop dispensers is in a designated alignment with respect to the rows and columns of features on substrate surface 11a. In FIG. 5, only a single row of features is shown for purposes of convenience. It is to be understood that an array normally has many more features as discussed herein below, which may be arranged, for example, in various rows and columns. The feature locations depicted are designated 26, 28, 30, 32, 34, 36, 38 and 40, respectively. First, drops of activator reagent are dispensed from dispenser 52a to predetermined locations on surface 11a of substrate 10 followed by dispensing of particular polymer forming reagents dA, dC, dG, dT. Accordingly, all of the nozzles of a drop dispenser that are to be triggered are triggered according to a nozzle firing pattern to deliver drops of a particular reagent to the surface of a substrate at specified feature locations. In the example shown, the nozzle firing pattern is as follows: nozzle 52a1 to feature location 26, nozzle 52b2 to feature location 28, nozzle 52d3 to feature location 30, nozzle 52c4 to feature location 32, nozzle 52b5 to feature location 34, nozzle 52d6 to feature location 36, nozzle 52d7 to feature location 38, nozzle 52c8 to feature location 40 in Swath 1 of drop dispensing device 20.

Then, substrate 10 is moved into a drop receiving relationship with drop dispensing device 60. It should be understood that the drop receiving relationship or the drop dispensing relationship between the substrate and the drop dispensing devices may be accomplished by moving the substrate relative to the drop dispensing device or moving the drop dispensing device with respect to the substrate or a combination thereof. In the example shown, the first column of nozzles of drop dispensing device 60 is aligned with feature 26 on substrate 10. It is, of course, within the scope of the present methods to align a column of nozzles other than the first column as long as a nozzle firing pattern is chosen to deposit drops of the particular reagents to the desired feature locations. In Swath 2, activator is not dispensed. Rather, another drop of nucleotide monomer reagent is delivered to a designated feature location. Accordingly, all of the nozzles of a drop dispenser to be triggered are triggered according to a selected nozzle firing pattern, i.e., the nozzle firing pattern for Swath 2, to deliver drops of a reagent to the surface of a substrate at specified feature locations. However, because of the change in the drop dispensing devices, a different nozzle, i.e., the nozzle of drop dispensing device 60, delivers the same reagent to a specified feature location that was to receive reagent in Swath 1. In the example shown, the nozzle firing pattern is as follows: nozzle 62a1 to feature location 26, nozzle 62b2 to feature location 28, nozzle 62d3 to feature location 30, nozzle 62c4 to feature location 32, nozzle 62b5 to feature location 34, nozzle 62d6 to feature location 36, nozzle 62d7 to feature location 38, nozzle 62c8 to feature location 40 in Swath 2 of drop dispensing device 60.

Although not shown in the figure, additional passes may be employed for a particular cycle utilizing drop dispensing devices 50 and 60 or additional drop dispensing devices. An appropriate nozzle firing pattern is implemented as discussed above to deliver reagents to specified feature locations on the surface of the substrate.

Following the dispensing of reagents for the dispensing step of the cycle in question, substrate 10 is placed in flow cell 42 where it is subjected to various treatment steps of the synthesis cycle such as, for example, blocking or capping, oxidation and deblocking or detritylation as mentioned above. In this embodiment the treatment is accomplished by treating the entire substrate (“flooding”) with a liquid layer of the appropriate reagent. The above protocol is repeated in a next cycle to deposit the appropriate polymer forming reagents for the next cycle in the synthesis. The cycles are repeated a sufficient number of times (cycle number) until the desired array of biopolymer features has been synthesized or until the array has been patterned. Final deprotection of nucleoside bases can be accomplished thereby producing the final array product.

### Protocol for Delivery of Activator Reagent Involving Two Different Nozzles of the Same Drop Dispenser or Two Different Nozzles of Two Different Drop Dispensers

In some embodiments the biopolymer forming reagents are dispensed for all of the cycles of the method until the array of biopolymers is prepared. For example, the biopolymer features may be polynucleotide features and the biopolymer forming reagents comprise nucleotide monomers and activators. In some embodiments at least the nucleotide monomers and activator are dispensed for the entire method until the array of biopolymers is prepared. In some embodiments at least the activator is dispensed for the entire method until the array of biopolymers is prepared. Any of the above mentioned protocols may be employed to carry out these approaches. That is, a protocol may be employed wherein the two different nozzles are on the same drop dispenser or a protocol wherein the two different nozzles are on two different drop dispensers.

An example of the above, by way of illustration and not limitation, is an embodiment wherein on the first pass or swath of the drop dispensing device, the drop dispenser is in a designated spatial relationship with respect to the rows and columns of features on the substrate surface. Nucleotide monomers are delivered to specified feature locations on a surface of a substrate using known protocols, i.e., using a single dispenser and nozzle firing pattern for all of the swaths of all of the cycles.

In accordance with the present methods, for the activation step of a cycle, all of the nozzles of a drop dispenser that are to be triggered for the synthesis cycle in question are triggered to deliver drops of activator reagent to the surface of a substrate at specified feature locations. The nozzles that are triggered are determined by the specified feature locations that are to receive a drop of activator as well as the chosen protocol and the nozzle firing pattern is determined by the pattern of the specified feature locations. Accordingly, a nozzle firing pattern is employed in the first pass of the activation step to deliver a drop of activator to the desired feature locations.

In accordance with embodiments of the present invention, in a second pass of the drop dispensing device for the activation step of the cycle in question, the drop dispenser is moved to change its spatial relationship with respect to the rows and columns of features as discussed above. As mentioned above, dispensing of nucleotide monomer reagents may be carried out according to known protocols, for example, where a single dispenser is employed for each of the monomer reagents and the nozzle firing pattern is the same as that used for delivery of drops of the nucleotide monomer reagents in the first pass of the cycle.

In the second pass of the cycle, an adjusted nozzle firing pattern is implemented for delivery of activator reagent so that a different nozzle delivers a drop of activator reagent to a feature location to which a drop of activator reagent was delivered in the first swath. The change in the nozzle firing pattern is implemented by appropriate software that enables a computer to communicate instructions to the drop dispensing device and the dispensers thereon. After the adjustment in spatial relationship of the drop dispenser, a second pass of the drop dispensing device is carried out for the activation step in the same cycle of the synthesis in which all of the nozzles of a drop dispenser to be triggered are triggered according to a nozzle firing pattern for the second pass to deliver drops of the same activator reagent to the surface of the substrate at specified feature locations so that a different nozzle delivers activator reagent to a feature location to which activator reagent was to be delivered to in the first pass. As a result, if one or more nozzles failed to deliver activator reagent to one or more feature locations in the first pass, the change in spatial relationship of the dispensing device with respect to the surface of the substrate in the second pass as well as the change in the nozzle firing pattern results in delivery of activator reagent to any feature locations that did not receive, but should have received, activator reagent on the first pass. This occurs because any nozzle that failed to deliver reagent is moved to a position such that the feature location receiving the activator reagent has already received a drop of reagent during the first pass. Likewise, a feature location that did not receive a drop of activator reagent during the first pass receives a drop of the reagent from a different, functioning nozzle on the second pass.

After the adjustment in orientation of the drop dispenser, and depending on the swath number for the particular cycle of the synthesis in question, a third pass of the drop dispensing device may or may not be carried out for the activation step of the same cycle of the synthesis. If a third pass is employed, all of the nozzles of a drop dispenser to be triggered are triggered according to an adjusted nozzle firing pattern to deliver drops of the same activator reagent to the surface of the substrate at specified feature locations. The nozzle firing pattern may be the same as, or different from, that of the first pass and/or the second pass. However, in accordance with the present embodiments, the feature locations for each swath are those that are to receive activator reagent for the activation step of the particular synthesis cycle. The passes may be continued in a similar manner until the swath number is achieved for a particular cycle of the synthesis. Changing the spatial relationship of the drop dispensing device to the feature locations on the surface of the substrate, as well as the corresponding nozzle firing pattern, for one, two, three, etc., up to all of the swaths of the cycle of the synthesis enhances the ability to avoid errors in the delivery of activator reagent to any one feature location.

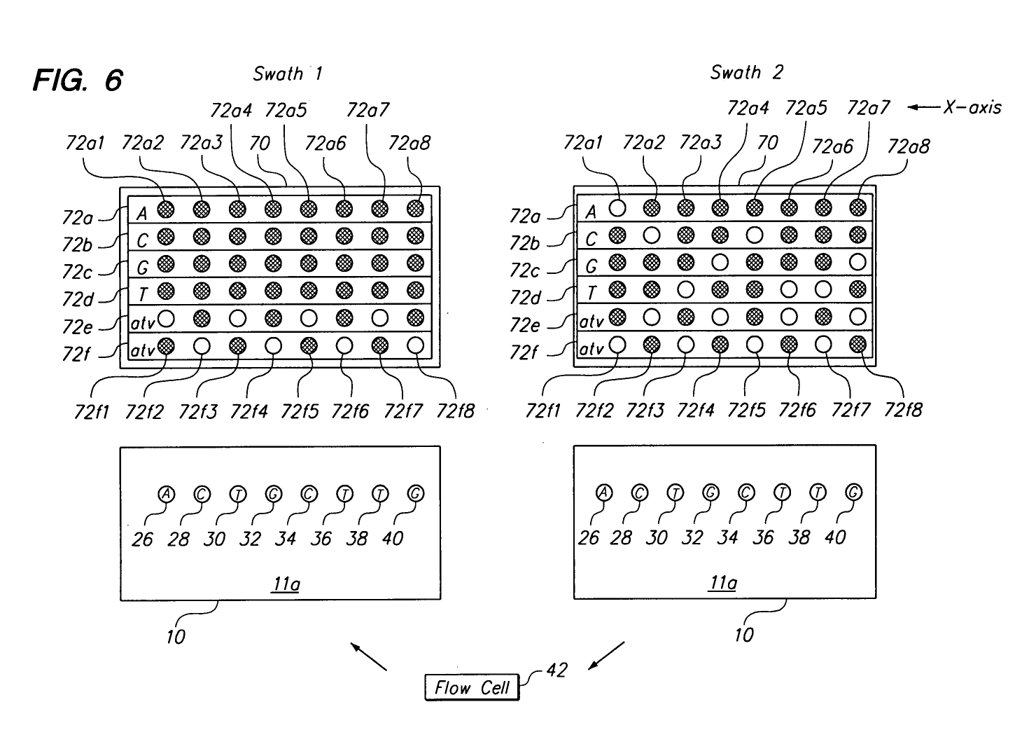

An illustrative example in accordance with the above embodiments is illustrated in FIG. 6 wherein two passes (Swath 1 and Swath 2) of a drop dispensing device 70 are depicted for the drop deposition step of a synthesis cycle. Drop dispensing device 70 is moved into a drop dispensing relationship with surface 11a of substrate 10. Drop dispensing device 70 is shown, for purposes of illustration and not limitation, with six drop dispensers 72a, 72b, 72c, 72d, 72e and 72f. Each of four drop dispensers dispenses a different nucleotide monomer reagent and two dispensers dispense an activator (atv), which may be, for example, tetrazole. The nucleotide monomer reagents are in the form of phosphoramidate reagents for synthesizing an array of polynucleotides on the surface of a substrate. Thus, as depicted, drop dispenser 72a dispenses nucleotide monomer dA, drop dispenser 72b dispenses nucleotide monomer dC, drop dispenser 72c dispenses nucleotide monomer dG, drop dispenser 72d dispenses nucleotide monomer dT and drop dispensers 72e and 72f dispense activator. Each of the dispensers comprises multiple orifices or nozzles, most of which are not shown. In FIG. 6, only a few of the nozzles for each of the drop dispensers are shown for purposes of convenience. The nozzles are designated by consecutive numbers, which follow the identifying designation for each of the drop dispensers. For example, the nozzles for drop dispenser 72a are designated 72a1, 72a2, 72a3, 72a4, 72a5, 72a6, 72a7, 72a8 and so forth.

On the first pass or Swath 1 of drop dispensing device 70 over substrate 10, each of the drop dispensers is in a designated alignment with respect to the rows and columns of features on substrate surface 11a. In FIG. 6, only a single row of features is shown for purposes of convenience. It is to be understood that an array normally has many more features as discussed herein below, which may be arranged, for example, in various rows and columns. The feature locations depicted are designated 26, 28, 30, 32, 34, 36, 38 and 40, respectively. In Swath 1, only drops of activator reagent are dispensed to predetermined locations on surface 11a of substrate 10. The drops of activator are dispensed from nozzles of drop dispensers 72e and 72f according to a preselected nozzle firing pattern. Accordingly, all of the nozzles of drop dispensers 72e and 72f that are to be triggered are triggered according to the preselected nozzle firing pattern to deliver drops of activator to the surface of a substrate at specified feature locations. In the example shown, the nozzle firing pattern is as follows: nozzle 72e1 to feature location 26, nozzle 72f2 to feature location 28, nozzle 72e3 to feature location 30, nozzle 72f4 to feature location 32, nozzle 72e5 to feature location 34, nozzle 72f6 to feature location 36, nozzle 72e7 to feature location 38, nozzle 72f8 to feature location 40 in Swath 1 of drop dispensing device 70. The polymer forming reagents dA, dC, dG, dT, respectively, are not dispensed in Swath 1.

Then, drop dispensing device 70 is employed in a second pass or swath where the alignment of the nozzles with respect to a column of feature locations is the same as the alignment of the nozzles for the first pass. In Swath 2, activator, as well as polymer forming reagents, is dispensed according to a second preselected nozzle firing pattern. Accordingly, all of the nozzles of a drop dispenser to be triggered are triggered according to an adjusted, or second, nozzle firing pattern, i.e., the nozzle firing pattern for Swath 2, to deliver drops of activator and polymer forming reagents to the surface of a substrate at specified feature locations. However, because of the change in the nozzle firing pattern, a different nozzle delivers activator to a specified feature location than the nozzle that was employed in Swath 1. In the example shown, the nozzle firing pattern for the activator is as follows: nozzle 72f1 to feature location 26, nozzle 72e2 to feature location 28, nozzle 72f3 to feature location 30, nozzle 72e4 to feature location 32, nozzle 72f5 to feature location 34, nozzle 72e6 to feature location 36, nozzle 72f7 to feature location 38, nozzle 72e8 to feature location 40 in Swath 1 of drop dispensing device 70. As a result, the feature locations receive activator from nozzles that are different between two different passes of a drop dispensing device.

During Swath 2, nucleotide monomers are also delivered to surface 11a of substrate 10. In the example shown, the nozzle firing pattern for the nucleotide monomer reagents is as follows: nozzle 72a1 to feature location 26, nozzle 72b2 to feature location 28, nozzle 72d3 to feature location 30, nozzle 72c4 to feature location 32, nozzle 72b5 to feature location 34, nozzle 72d6 to feature location 36, nozzle 72d7 to feature location 38, nozzle 72c8 to feature location 40 in Swath 2 of drop dispensing device 20.

Although not shown in the figure, the drop dispensing device may be moved again to deposit activator prior to depositing nucleotide monomer reagents. In such a situation, the nozzle firing pattern employed in Swath 1 for the activator may be employed or other nozzle firing patterns may be utilized.

Following the dispensing of reagents for the dispensing step of the cycle in question, substrate 10 is placed in flow cell 42 where it is subjected to various treatment steps of the synthesis cycle such as, for example, blocking or capping, oxidation and deblocking or detritylation as mentioned above. In this embodiment the treatment is accomplished by treating the entire substrate (“flooding”) with a liquid layer of the appropriate reagent. The above protocol is repeated in a next cycle to deposit the appropriate polymer forming reagents for the next cycle in the synthesis. In accordance with this embodiment, activator is deposited according to the aforementioned protocol. However, deposition of nucleotide monomer reagents is carried out according to the same nozzle firing pattern as employed in the previous cycle. The cycles are repeated a sufficient number of times (cycle number) until the desired array of biopolymer features has been synthesized or until the array has been patterned. Final deprotection of nucleoside bases can be accomplished thereby producing the final array product.

### Protocol for Patterning an Array Involving Two Different Nozzles on Two Different Drop Dispensers

In some embodiments the biopolymer forming reagents are dispensed for cycles of the method until the array of biopolymers is patterned. For example, the biopolymer features may be polynucleotide features and the biopolymer forming reagents comprise nucleotide monomers and activators. In some embodiments at least the nucleotide monomers and activator are dispensed for a portion of the cycles until the array of polynucleotides is patterned. Any of the above mentioned protocols may be employed to carry out these approaches. That is, a protocol may be employed wherein the two different nozzles are on the same drop dispenser or a protocol wherein the two different nozzles are on two different drop dispensers.

An example of the above, by way of illustration and not limitation, is an embodiment wherein on the first pass or swath of the drop dispensing device, one drop dispenser (arbitrarily designated as a first drop dispenser) is in a designated spatial relationship with respect to rows and columns of features on the substrate surface. All of the nozzles of a drop dispenser that are to be triggered for the synthesis cycle in question are triggered to deliver drops of a reagent to the surface of a substrate at specified feature locations. The nozzles that are triggered are determined by the specified feature locations that are to receive a drop of reagent as well as the chosen protocol. Accordingly, a nozzle firing pattern is employed in the first pass to deliver a drop of reagent to each of the desired feature locations.

In accordance with embodiments of the present invention, another drop dispenser (arbitrarily designated as a second drop dispenser) is present on the same dispensing device. During the first pass or swath of the second drop dispenser is in a designated spatial relationship with respect to the rows and columns of features on the substrate surface so that the second dispenser can dispense the same reagent, as that dispensed by the first dispenser, from selected nozzles to the same locations as those to which the first dispenser dispensed. All of the nozzles of the second drop dispenser that are to be triggered for the synthesis cycle in question are triggered to deliver drops of a reagent from the second dispenser to the surface of a substrate at specified feature locations. The nozzles that are triggered are determined by the specified feature locations that are to receive a drop of reagent as well as the chosen protocol. Accordingly, a nozzle firing pattern is employed for the second dispenser in the first pass to deliver a drop of reagent to the desired feature locations. The control of the nozzle firing pattern for the first and the second dispensers is implemented by appropriate software that enables a computer to communicate instructions to the drop dispensing device.

Cycles employing the above protocol are carried out until an array of features is patterned. Once patterning of the array is achieved, redundant dispensers are discontinued and large feature sizes are obtained with smaller volumes of reagents applied according to known protocols. In accordance with the above example, after the array of features is patterned, only one dispenser is used to dispensed a particular reagent and a single nozzle firing pattern is used for the cycle in question. The cycles are repeated a sufficient number of times to prepare the array of polynucleotide features or until the array has been patterned.

### Discussion of Polynucleotide Synthesis

One in situ method employs inkjet printing technology to dispense appropriate phosphoramidite reagents and other reagents necessary for forming the polynucleotide onto individual sites on a surface of a substrate. Oligonucleotides are synthesized on a surface of a substrate in situ using phosphoramidite chemistry. Solutions containing nucleotide monomers and other reagents as necessary such as an activator, e.g., tetrazole, are applied to the surface of a substrate by means of, for example, piezo ink-jet technology or thermal ink-jet technology. Individual drops of reagents are applied to reactive areas on the surface using a piezo ink-jet type nozzle or a thermal ink-jet type nozzle.

The surface of the substrate may have an alkyl bromide trichlorosilane coating to which is attached polyethylene glycol to provide terminal hydroxyl groups. These hydroxyl groups provide for linking to a terminal primary amine group on a monomeric reagent. Excess of non-reacted chemical on the surface is washed away in a subsequent step.

Following the dispensing step of a synthetic cycle, the surface is subjected to reagents to prepare the surface for repeating the drop-dispensing step. The above steps of dispensing reagents and preparing the surface of each cycle are repeated a sufficient number of times to synthesize the array of polymeric compounds. The nature of the reagents to prepare the surface for the next step is dependent on the nature of the polymers that are formed, on the nature of the polymer forming reagents and the synthetic procedure employed, and the like. For in situ fabrication methods, in many embodiments multiple different reagent droplets are deposited on the surface of a substrate at a given target location in order to form the final feature or polymer at that location. The in situ fabrication methods include those described in U.S. Pat. No. 5,449,754 for synthesizing peptide arrays, and in U.S. Pat. No. 6,180,351 and WO 98/41531 and the references cited therein for polynucleotides.

For example, an in situ method for fabricating a polynucleotide array typically follows, at each of the multiple different locations or addresses at which polymer features are to be formed, the same conventional iterative sequence used in forming polynucleotides from nucleoside reagents on a substrate by means of known chemistry. This iterative sequence can be considered as multiple ones of the following attachment cycle at each feature to be formed: (a) coupling an activated selected nucleoside (a monomeric unit) through a phosphite linkage to a functionalized substrate in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the blocking group such as a trityl group (“deblocking”) from the now substrate bound nucleoside coupled in step (a), to generate a reactive site for the next cycle of these steps. In the above method, the coupling can be performed, for example, by depositing drops of an activator and phosphoramidite at the specific desired feature locations for the array. Capping, oxidation and deblocking or detritylation can be accomplished by treating the entire substrate (“flooding”) with a layer of the appropriate reagent. The functionalized substrate (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in another flooding procedure in a known manner. Activator may be dispensed utilizing a drop dispensing device.

The foregoing chemistry of the synthesis of polynucleotides is described in detail, for example, in Caruthers, Science 230: 281-285, 1985; Itakura, et al., Ann. Rev. Biochem. 53: 323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, and 5,869,643, EP 0294196, and elsewhere.

An “array” includes any one-, two- or three-dimensional arrangement of addressable regions bearing a particular feature such as a biopolymer, e.g., polynucleotides, associated with that region. An array is addressable in that it has multiple regions of different moieties, for example, different polynucleotide sequences, such that a region or feature or spot of the array at a particular predetermined location or address on the array can detect a particular target molecule or class of target molecules although a feature may incidentally detect non-target molecules of that feature.

An array assembly on the surface of a substrate refers to one or more arrays disposed along a surface of an individual substrate and separated by inter-array areas. Normally, the surface of the substrate opposite the surface with the arrays (opposing surface) does not carry any arrays. The arrays can be designed for testing against any type of sample, whether a trial sample, a reference sample, a combination of the foregoing, or a known mixture of components such as polynucleotides, proteins, polysaccharides and the like (in which case the arrays may be composed of features carrying unknown sequences to be evaluated). The surface of the substrate may carry at least one, two, four, or at least ten, arrays. Depending upon intended use, any or all of the arrays may be the same or different from one another and each may contain multiple spots or features of chemical compounds such as, e.g., biopolymers in the form of polynucleotides or other biopolymer.

As mentioned above, an individual substrate may contain a single array or multiple arrays. Features of the array may be arranged in rectilinear rows and columns. This is particularly attractive for single arrays on a substrate. When multiple arrays are present, such arrays can be arranged, for example, in a sequence of curvilinear rows across the substrate surface (for instance, a sequence of concentric circles or semi-circles of spots), and the like.

Each feature, or element, within the molecular array is defined to be a small, regularly shaped region of the surface of the substrate. The features are arranged in a predetermined manner. Each feature of an array usually carries a predetermined biopolymer or mixtures thereof. Each feature within the molecular array may contain a different molecular species, and the molecular species within a given feature may differ from the molecular species within the remaining features of the molecular array. Some or all of the features may be of different compositions. Each array may contain multiple spots or features and each array may be separated by spaces or areas. It will also be appreciated that there need not be any space separating arrays from one another. Interarray areas and interfeature areas are usually present but are not essential. The interarray and interfeature areas do not carry any polynucleotide (or other biopolymer of a type of which the features are composed). Interarray areas and interfeature areas typically will be present where arrays are formed by the conventional in situ process by depositing for each feature at least one droplet of reagent such as from a pulse jet. It will be appreciated though that the interarray areas and interfeature areas, when present, could be of various sizes and configurations as discussed below.

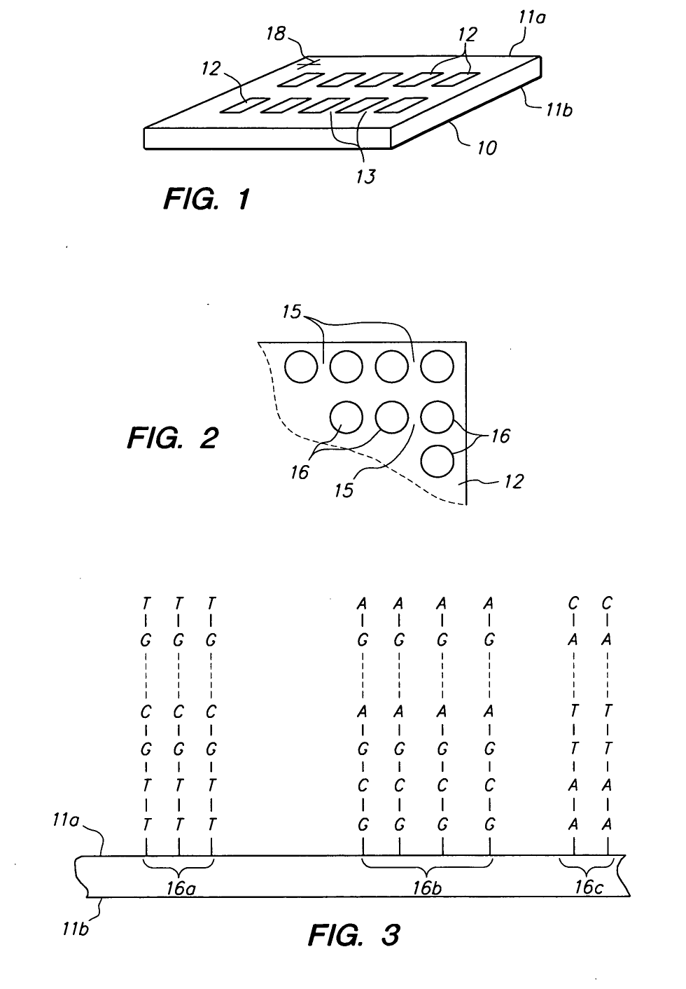

Referring to FIGS. 1-3, multiple identical arrays 12 (only some of which are shown in FIG. 1) are shown separated by inter-array regions 13, across the complete front surface 11a of a single transparent substrate 10, which also includes rear surface 11b. Front surface 11a includes fiducial marking 18. However, the arrays 12 produced on a given substrate need not be identical and some or all could be different. Each array 12 will contain multiple spots or features 16 (16a, 16b, 16c, etc.) separated by inter-feature regions 15. A typical array 12 may contain from 100 to 500,000 or more features. All of the features 16 may be different, or some or all may be the same. Each feature carries a predetermined moiety (such as a particular polynucleotide sequence), or a predetermined mixture of moieties (such as a mixture of particular polynucleotides). This is illustrated schematically in FIG. 3 where different regions 16 are shown as carrying different polynucleotide sequences.

As referred to above, embodiments of the invention have particular application to substrates bearing oligomers or polymers. The oligomer or polymer is a chemical entity that contains a plurality of monomers. It is generally accepted that the term “oligomers” is used to refer to a species of polymers. The terms “oligomer” and “polymer” may be used interchangeably herein. Polymers usually comprise at least two monomers. Oligomers generally comprise about 6 to about 20,000 monomers, preferably, about 10 to about 10,000, more preferably about 15 to about 4,000 monomers. Examples of polymers include polydeoxyribonucleotides, polyribonucleotides, other polynucleotides that are C-glycosides of a purine or pyrimidine base, or other modified polynucleotides, polypeptides, polysaccharides, and other chemical entities that contain repeating units of like chemical structure. Exemplary of oligomers are oligonucleotides and oligopeptides. It is important to note that some skilled in the art classify oligonucleotides as containing less than a specified number of nucleotides such as 100 or less nucleotides and classify polynucleotides as containing more than a specified number of nucleotides such as more than 100 nucleotides. As used herein, the term polynucleotide includes oligonucleotides.

The present methods have particular application to the preparation of arrays comprising biopolymers. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups such as, for example, poly peptide-nucleic acid analogs. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions.

Polynucleotides are compounds or compositions that are polymeric nucleotides or nucleic acid polymers. The polynucleotide may be a natural compound or a synthetic compound. Polynucleotides include oligonucleotides and are comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also used. The polynucleotide can have from about 2 to 5,000,000 or more nucleotides. Usually, the oligonucleotides are at least about 2 nucleotides, usually, about 5 to about 100 nucleotides, more usually, about 10 to about 50 nucleotides, and may be about 15 to about 30 nucleotides, in length. Polynucleotides include single or multiple stranded configurations, where one or more of the strands may or may not be completely aligned with another.

A nucleotide refers to a sub-unit of a nucleic acid and has a phosphate group, a 5 carbon sugar ring and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, the term “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides.

Preferred materials for the substrate on which the synthesis takes place are those materials that provide physical support for the chemical compounds that are deposited on the surface or synthesized on the surface in situ from subunits. The materials should be of such a composition that they endure the conditions of a deposition process and/or an in situ synthesis and of any subsequent treatment or handling or processing that may be encountered in the use of the particular array.

Typically, the substrate material is transparent. By “transparent” is meant that the substrate material permits signal from features on the surface of the substrate to pass therethrough without substantial attenuation and also permits any interrogating radiation to pass therethrough without substantial attenuation. By “without substantial attenuation” may include, for example, without a loss of more than 40% or more preferably without a loss of more than 30%, 20% or 10%, of signal. The interrogating radiation and signal may for example be visible, ultraviolet or infrared light. In certain embodiments, such as for example where production of binding pair arrays for use in research and related applications is desired, the materials from which the substrate may be fabricated should ideally exhibit a low level of non-specific binding during hybridization events. However, it should be noted that the nature of the transparency of the substrate is somewhat dependent on the nature of the scanner employed to read the substrate surface. Some scanners work with opaque or reflective substrates.

The materials may be naturally occurring or synthetic or modified naturally occurring. Suitable rigid substrates may include glass, which term is used to include silica, and include, for example, glass such as glass available as Bioglass, and suitable plastics. Should a front array location be used, additional rigid, non-transparent materials may be considered, such as silicon, mirrored surfaces, laminates, ceramics, opaque plastics, such as, for example, polymers such as, e.g., poly (vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc., either used by themselves or in conjunction with other materials. The surface of the substrate is usually the outer portion of a substrate.

The surface of the material onto which the chemical compounds are deposited or formed may be smooth and/or substantially planar, or have irregularities, such as depressions or elevations. The surface may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethylene amines, polyarylene sulfides, polysiloxanes, polyimides, polyacetates, and the like, where the polymers may be hetero- or homo-polymeric, and may or may not have separate functional moieties attached thereto (for example, conjugated). Various further modifications to the particular embodiments described above are, of course, possible. Accordingly, the present invention is not limited to the particular embodiments described in detail above.

The material used for an array substrate or substrate may take any of a variety of configurations ranging from simple to complex. Usually, the material is substantially rectangular and relatively planar such as, for example, a slide. In many embodiments, the material is shaped generally as a rectangular solid. As mentioned above, multiple arrays of chemical compounds are synthesized on a sheet, which is then singulated, such as, e.g., cut by breaking along score lines, into single array slides. The sheet of material may be of any convenient size depending on the nature of the equipment used, production lot size, production efficiencies, production throughput demands, and so forth. In some embodiments, the sheet of material is usually about 5 to about 13 inches in length and about 5 to about 13 inches in width so that the sheet may be divided into multiple single array substrates having the dimensions indicated below. The thickness of the substrate is about 0.01 mm to 5.0 mm, usually from about 0.1 mm to 2 mm and more usually from about 0.2 to 1. In a specific embodiment by way of illustration and not limitation, a wafer that is 6.25 inches by 6 inches by 1 mm is employed.

The surface of a substrate is normally treated to create a primed or functionalized surface, that is, a surface that is able to substrate the attachment of a fully formed chemical compound or the synthetic steps involved in the production of the chemical compound on the surface of the substrate. Functionalization relates to modification of the surface of a substrate to provide a plurality of functional groups on the substrate surface. By the term “functionalized surface” is meant a substrate surface that has been modified so that a plurality of functional groups are present thereon usually at discrete sites on the surface. The manner of treatment is dependent on the nature of the chemical compound to be synthesized and on the nature of the substrate surface. In one approach a reactive hydrophilic site or reactive hydrophilic group is introduced onto the surface of the substrate. Such hydrophilic moieties can be used as the starting point in a synthetic organic process.

In one embodiment, the surface of the substrate, such as a glass substrate, is siliceous, i.e., the surface comprises silicon oxide groups, either present in the natural state, e.g., glass, silica, silicon with an oxide layer, etc., or introduced by techniques well known in the art. One technique for introducing siloxyl groups onto the surface involves reactive hydrophilic moieties on the surface. These moieties are typically epoxide groups, carboxyl groups, thiol groups, and/or substituted or unsubstituted amino groups as well as a functionality that may be used to introduce such a group such as, for example, an olefin that may be converted to a hydroxyl group by means well known in the art. One approach is disclosed in U.S. Pat. No. 5,474,796 (Brennan), the relevant portions of which are incorporated herein by reference. A siliceous surface may be used to form silyl linkages, i.e., linkages that involve silicon atoms. Usually, the silyl linkage involves a silicon-oxygen bond, a silicon-halogen bond, a silicon-nitrogen bond, or a silicon-carbon bond.

Another method for attachment is described in U.S. Pat. No. 6,219,674 (Fulcrand, et al.). A surface is employed that comprises a linking group consisting of a first portion comprising a hydrocarbon chain, optionally substituted, and a second portion comprising an alkylene oxide or an alkylene imine wherein the alkylene is optionally substituted. One end of the first portion is attached to the surface and one end of the second portion is attached to the other end of the first portion chain by means of an amine or an oxy functionality. The second portion terminates in an amine or a hydroxy functionality. The surface is reacted with the substance to be immobilized under conditions for attachment of the substance to the surface by means of the linking group.

Another method for attachment is described in U.S. Pat. No. 6,258,454 (Lefkowitz, et al.). A solid substrate having hydrophilic moieties on its surface is treated with a derivatizing composition containing a mixture of silanes. A first silane provides the desired reduction in surface energy, while the second silane enables functionalization with molecular moieties of interest, such as small molecules, initial monomers to be used in the solid phase synthesis of oligomers, or intact oligomers. Molecular moieties of interest may be attached through cleavable sites.

A procedure for the derivatization of a metal oxide surface uses an aminoalkyl silane derivative, e.g., trialkoxy 3-aminopropylsilane such as aminopropyltriethoxy silane (APS), 4-aminobutyltrimethoxysilane, 4-aminobutyltriethoxysilane, 2-aminoethyltriethoxysilane, and the like. APS reacts readily with the oxide and/or siloxyl groups on metal and silicon surfaces. APS provides primary amine groups that may be used to carry out the present methods. Such a derivatization procedure is described in EP 0 173 356 B1, the relevant portions of which are incorporated herein by reference. Other methods for treating the surface of a substrate will be suggested to those skilled in the art in view of the teaching herein.

The devices and methods of the present invention are particularly useful for the preparation of individual substrates with an array of biopolymers. An array includes any one-, two- or three-dimensional arrangement of addressable regions bearing a particular biopolymer such as polynucleotides, associated with that region. An array is addressable in that it has multiple regions of different moieties, for example, different polynucleotide sequences, such that a region or feature or spot of the array at a particular predetermined location or address on the array can detect a particular target molecule or class of target molecules although a feature may incidentally detect non-target molecules of that feature.

Normally, the surface of the substrate opposite the surface with the array (opposing surface) does not carry any arrays. The arrays can be designed for testing against any type of sample, whether a trial sample, a reference sample, a combination of the foregoing, or a known mixture of components such as polynucleotides, proteins, polysaccharides and the like (in which case the arrays may be composed of features carrying unknown sequences to be evaluated).

Any of a variety of geometries of arrays on a substrate may be used. As mentioned above, an individual substrate usually contains a single array but in certain circumstances may contain more than one array. Features of the array may be arranged in rectilinear rows and columns. This is particularly attractive for single arrays on a substrate. The configuration of the arrays and their features may be selected according to manufacturing, handling, and use considerations.

Regardless of the geometry of the array on the surface of an individual substrate or on the surface of a sheet comprising a multiple of individual substrates, the arrays normally do not comprise the entire surface of the sheet or of the substrate. For sheets of material comprising a multiple of individual substrates, the sheet typically has a border along its longitudinal edges that is about 0.5 to about 3 mm wide, usually, about 1 to about 2 mm wide. In many embodiments, the border of the individual substrates obtained from the sheet has the same dimensions as the border for the sheet. In some embodiments one area of the individual substrate that is a non-interfeature area or a portion of a border or a combination thereof comprises an identifier such as, e.g., a bar code. It is often desirable to have some type of identification on the array substrate that allows matching a particular array to layout information, since array layout information in some form is used to meaningfully interpret the information obtained from interrogating the array.

As mentioned above, the surface of an individual substrate may have only one array or more than one array. Depending upon intended use, the array may contain multiple spots or features of chemical compounds such as, e.g., biopolymers in the form of polynucleotides or other biopolymer. A typical array on an individual substrate may contain more than ten, more than one hundred, more than five hundred, more than one thousand, more than fifteen hundred, more than two thousand, more than twenty five hundred features, more than 20,000, more than 25,000, more than 30,000, more than 35,000, more than 40,000, more than 50,000, more than 75,000, or more than 100,000 features. In many embodiments the number of features on the individual substrates is in the range of about 100 to about 100,000, about 1000 to about 100,000 and so forth. The features may occupy an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges.

Each feature, or element, within the molecular array is defined to be a small, regularly shaped region of the surface of the substrate. The features are arranged in a predetermined manner. Each feature of an array usually carries a predetermined chemical compound or mixtures thereof. Each feature within the molecular array may contain a different molecular species, and the molecular species within a given feature may differ from the molecular species within the remaining features of the molecular array. Some or all of the features may be of different compositions. Each array may contain multiple spots or features separated by spaces or areas that have no features. Such interfeature areas are usually present but are not essential. As with the border areas discussed above, these interfeature areas do not carry any chemical compound such as polynucleotide (or other biopolymer of a type of which the features are composed). Interfeature areas typically will be present where arrays are formed by deposition of polymer subunits, as described above. It will be appreciated though that the interfeature areas, when present, could be of various sizes and configurations.

Following the dispensing of reagents for a particular cycle, the substrates may be placed into respective flow cells where they are subjected to various treatment steps such as, for example, capping, oxidation and deblocking or detritylation as mentioned above. In this embodiment the treatment is accomplished by treating the entire substrate (“flooding”) with a liquid layer of the appropriate reagent. The above protocol is repeated in a subsequent cycle to deposit the appropriate polymer forming reagents for the next cycle in the synthesis. The cycles or steps are repeated until the desired arrays have been synthesized. Final deprotection of nucleoside bases can be accomplished as discussed above thereby producing the final array product.

### Embodiments of Apparatus

The phrase “adapted to” or “adapted for” is used herein with respect to components of the present apparatus. The components of the present apparatus are adapted to perform a specified function by a combination of hardware and software. This includes the structure of the particular component and may also, and usually does, include a microprocessor, embedded real-time software and I/O interface electronics to control the sequence of operations of the invention.

Some embodiments of the present invention are directed to apparatus for preparing an array of polymeric compounds on a substrate from multiple polymer subunits. In some embodiments the invention is directed to an apparatus for preparing an array of biopolymer features on a surface of a substrate. The apparatus comprises (a) at least one drop dispensing device comprising one or more drop dispensers each comprising multiple nozzles, (b) a mechanism for moving the drop dispensing device or a substrate relative to one another in one or more drop dispensing cycles to dispense biopolymer forming reagents to the surface of the substrate, and (c) a computer comprising a computer program to control the movement of the mechanism and the activation of the nozzles of the drop dispensing device in a protocol such that, for at least one of the biopolymer forming reagents and for at least a portion of the method, drops of reagents are dispensed to feature locations from at least two different nozzles for each reagent in at least two different passes of a drop dispensing device. The computer program may also control movement of the mechanism and the activation of the nozzles of the drop dispensing device to dispense single drops of biopolymer forming reagent of predetermined volume.

In some embodiments the apparatus comprise a drop dispensing device moving mechanism adapted to move the drop dispensing device relative to a surface of a substrate on a substrate mount to bring the drop dispensing device into drop dispensing relationship with the surface. In some embodiments, the apparatus comprises a substrate mount and a substrate moving mechanism adapted to move the substrate to a processing station and back to the substrate mount. In some embodiments the apparatus comprises a substrate mount moving mechanism for moving the substrate into a drop dispensing relationship with the drop dispensing device. In some embodiments both the substrate mount moving mechanism and the drop dispensing device moving mechanism are adapted to move relative to one another to establish a drop dispensing relationship with the surface of the substrate.

The moving mechanisms are generally automated devices. Such automated devices comprise at least a means for precisely controlling the position of the drop-dispensing device with respect to a substrate surface. Examples of such means include, for example, an XYZ translational mechanism, e.g., an XYZ translational arm to which the drop dispensing device is rigidly fixed. In some embodiments the moving mechanism also comprises means for firing the head. Such automated devices are well known to those of skill in the printing and document production art.

In some embodiments the moving mechanism is adapted for moving a drop dispensing device or a substrate mount for translation along an x-axis and/or a y-axis and/or a z-axis. The movement of a drop dispensing device mount may be independent of the movement of the substrate mount along the respective axes, e.g., a y-axis. Translation along an x-axis provides for moving the dispensing device transversely to the direction of movement of the substrate mount (along the y-axis) and in position for dispensing of reagents to the surface of a substrate. In one approach the drop dispensing device is carried by a stage arrangement, which provides for the desired movement parameters. In this approach the dispensing device is secured to the stage, which is usually attached to a frame member of an apparatus. For example, in one approach the drop dispensing device may be carried by an orthogonal z-axis stage arrangement attached to an x-axis stage arrangement, which is attached directly to a rigid supporting beam off a base to which the substrate mount is secured. Other approaches for providing the dispensing device with desired movement capabilities may be employed.

To achieve the desired level of dispensing accuracy, the substrate on the substrate mount should be oriented parallel to dispensing device on the y-axis. The positioning of the substrate mount relative to the dispensing device is accomplished using optical systems, which comprise at least one, and in some optical systems, more than one image sensor. Usually, an optical system is employed for positioning the substrate mount along the y-axis as described above. In this instance the optical system usually comprises at least two image sensors. An optical system is employed for positioning the dispensing device along the x-axis. In this instance the optical system usually comprises at least one image sensor. Thus, the optical systems are cooperative to position the dispensing device and the substrate mount relative to one another. Usually, the image sensor is part of a camera.

In some embodiments the components of the apparatus may be mounted on a suitable frame in a manner consistent with the present invention. The frame of the apparatus is generally constructed from a suitable material that gives structural strength to the apparatus so that various moving parts may be employed in conjunction with the apparatus. Such materials for the frame include, for example, metal, lightweight composites, granite and the like.

The apparatus, in some embodiments, may comprise a loading station for loading reagents into the dispensing device and a mechanism for moving the dispensing device and/or the loading station relative to one another. In some embodiments, the apparatus may also comprise a cleaning station or a washing station for cleaning or washing the dispensing device or surfaces of the dispensing device and a mechanism for moving the dispensing device and/or the cleaning or washing station relative to one another. In some embodiments the apparatus further may comprise a mechanism for inspecting the reagent deposited on the surface of the substrate.

The substrate mount may be any convenient structure on which the substrate may be placed and held for depositing reagents on the surface of the substrate. The substrate mount may be of any size and shape and generally has a shape similar to that of the substrate as long as it is sufficiently able to support the substrate. For example, the substrate mount may be rectangular for a rectangular substrate, circular for a circular substrate and so forth. The substrate mount may be constructed from any material of sufficient strength to physically receive and hold the substrate during the deposition of reagents on the substrate surface as well as to withstand the rigors of movement in one or more directions. Such materials include metals, plastics, composites, and the like.

The substrate may be retained on the substrate mount by gravity, friction, vacuum, and the like. The surface of the substrate mount, on which the substrate is received, may be flat. On the other hand, the surface of the substrate mount may comprise certain structural features such as, for example, parallel upstanding linear ribs, and the like, on which the substrate is placed. Whether the substrate mount is flat or comprises structural features, the resulting surface of the substrate mount on which the substrate rests is planar. The nature and number of structural features is generally determined by the size, weight and shape of the substrate, and so forth. In one embodiment the upper surface of the substrate mount has openings that communicate with a suitable vacuum source to hold the substrate on the substrate mount. The openings may be in the surface of the substrate mount or in structural features on the surface of the substrate mount. In a specific embodiment the substrate mount is a vacuum chuck.

In some embodiments the substrate mount is adapted for movement along certain axes such as, for example, translation along a y-axis and/or for rotation about a center axis that is parallel to a z-axis. Translation along a y-axis provides for moving a substrate on the substrate mount in position for dispensing of reagents to a surface of the substrate. Usually, this requires that the surface of the substrate be parallel to the surface of the dispensing device on which dispensing nozzles are located. Accordingly, the surface of the substrate is normal to the direction in which fluid is dispensed to the surface of the substrate. The ability of the substrate to rotate about a central axis allows any optical system, as discussed below, associated with the substrate mount to provide accurate orientation of the substrate with respect to a dispensing device during the dispensing of reagents to the surface of the substrate.

In one exemplary approach the substrate mount is carried by a stage arrangement, which provides for the desired movement parameters independently of the movement of the dispensing device. In this approach the substrate mount is secured to the stage, which is usually attached to a frame member of the apparatus. For example, the substrate mount may be carried by a stacked Increment-Theta stage arrangement that is attached directly to a granite base. Other approaches for providing the substrate mount with desired movement capabilities may be employed.

In some embodiments the drop dispensing device is adapted for translation along an x-axis independently of the movement of the substrate mount along the y-axis. Translation along an x-axis provides for moving the drop-dispensing device transversely to the direction of movement of the substrate mount (along the y-axis) and in position for dispensing of reagents to the surface of a substrate. In one approach the drop dispensing device is carried by a stage arrangement, which provides for the desired movement parameters. In this approach the drop dispensing device is secured to the stage, which is usually attached to a frame member of the present apparatus. For example, in one approach the drop dispensing device may be carried by an orthogonal z-axis stage arrangement attached to an x-axis stage arrangement, which is attached directly to a rigid supporting granite beam off a granite base to which the substrate mount is secured. Other approaches for providing the drop-dispensing device with desired movement capabilities may be employed.

In some embodiments to achieve the desired level of dispensing accuracy, the substrate on the substrate mount is oriented parallel to dispensing device on the y-axis. In some embodiments positioning of the substrate mount relative to the dispensing device is accomplished using optical systems, which comprise at least one, and in some optical systems, more than one image sensor. Usually, an optical system is employed for positioning the substrate mount along the y-axis as described above. Usually, the image sensor is part of a camera.

In some embodiments the present apparatus may also comprise a delivery device for delivering the substrate to the substrate mount. The delivery device has the function of receiving or removing a substrate from a substrate supply device and transporting the substrate to the substrate mount. Thus, the delivery device may have any convenient configuration, as long it is able to carry out the above functions. In one embodiment the delivery device is in the form of a two-prong fork where the supporting members (or prongs) of the fork are adapted to receive and carry the substrate. Usually, the prongs are designed to engage the underside surface of the substrate at the perimeter of the substrate. The delivery device may be made of any material that has the structural strength to carry the substrate and withstand the transport functions of the delivery device. Such materials include, for example, metals, lightweight composites, and so forth. The substrate may be retained on the substrate mount by gravity, friction, vacuum, and the like. In one embodiment the upper surface of the substrate mount has openings that communicate with a suitable vacuum source to hold the substrate on the substrate mount. The openings may be in the surface of the substrate mount or in structural features or support members on the surface of the substrate mount.

Another function of the delivery device is to deliver the substrate to the substrate mount so that preliminary adjustments may be made to provide the substrate to the substrate mount in a desired predetermined orientation. In this way the optical system of the substrate mount needs only to fine tune the orientation thereby achieving the desired predetermined orientation of the substrate relative to the dispensing device. To this end, the delivery device has associated therewith a delivery device optical system for positioning the substrate along an x-axis and a y-axis. The optical system may be similar in design to that discussed above for the substrate mount optical system. Thus, the delivery device optical system may comprise at least one image sensor. The delivery device is capable of translation along an x-axis and a y-axis and also is rotatable about a center axis so that the image sensors may communicate to a computer, which in turn may communicate with a mechanism such as a motor and the like that is responsible for the movement of the delivery device, to correct for deviations from the predetermined orientation for the substrate on the delivery device. Other configurations for the delivery device may also be employed.

In some embodiments the apparatus of the present invention may also comprise a loading station for loading reagents into the drop dispensing device. The loading station may be positioned in the present apparatus in a manner similar to that of a cleaning or washing station. Accordingly, the loading station may be placed in line with a cleaning or washing station so that it moves transversely with respect to the drop-dispensing device, which moves on the x-axis. The loading station may be of any convenient structure as long as the function of filling the dispensers of the drop-dispensing device with reagents to be dispensed is accomplished. The loading station comprises appropriate controls for controlling the temperature, humidity and the like of the components of the loading station including the reagents contained therein. The loading station also comprises appropriate circuitry and motors for controlling the movement of the loading station parallel to the x-axis. An example of an embodiment of a suitable loading station, by way of illustration and not limitation is described in U.S. Pat. No. 6,689,323, the relevant disclosure of which is incorporated by reference.

In some embodiments the present apparatus may also comprise a mechanism and method for accurately and rapidly observing deposition of droplets of liquid on the surface of a substrate. One such mechanism and method is described in U.S. Pat. No. 6,232,072 B1, issued May 15, 2001 (Fisher). The method includes depositing droplets of fluid carrying a biopolymer or a biomonomer on a front side of a transparent substrate. Light is directed through the substrate from the front side, back through a substrate back side and a first set of deposited droplets on the first side to an image sensor. In this manner, the first set is “imaged”.

In some embodiments the apparatus may also comprise a cleaning or washing station. Depending on the nature of the dispensers, this cleaning or washing may involve wiping the nozzle area of the dispensers or may involve a washing of the nozzle area and/or the dispensers.

As mentioned above, the apparatus and the methods in accordance with the present invention may be automated. To this end the apparatus of the invention further comprises appropriate motors and electrical and mechanical architecture and electrical connections, wiring and devices such as timers, clocks, computers and so forth for operating the various elements of the apparatus. Such architecture is familiar to those skilled in the art and will not be discussed in more detail herein.

To assist in the automation of the present process, the functions and methods may be carried out under computer control, that is, with the aid of a computer and computer program. For example, an IBM® compatible personal computer (PC) may be utilized. The computer is driven by software specific to the methods described herein. Software that may be used to carry out the methods may be, for example, Microsoft Excel or Microsoft Access and the like, suitably extended via user-written functions and templates, and linked when necessary to stand-alone programs that perform other functions.

Another aspect of embodiments of the present invention is a computer program product comprising a computer readable storage medium having a computer program stored thereon which, when loaded into a computer, performs the aforementioned method and/or controls the functions of the aforementioned apparatus. In particular, the computer program controls the movement of the mechanism and the activation of the orifices of a drop dispenser of the drop dispensing device in a protocol such that, for at least one of the biopolymer forming reagents and for at least a portion of the method, drops of reagents are dispensed to feature locations from at least two different orifices for each reagent in at least two different passes of a drop dispensing device.

In some embodiments the computer program controls the activation of the orifices such that the two different orifices are on the same drop dispenser.

In some embodiments the apparatus comprises at least two different drop dispensers for each reagent dispensed and the computer program controls the activation of the orifices such that the at least two different orifices are on the at least two different drop dispensers.

In some embodiments the biopolymer features are polynucleotide features and the biopolymer forming reagents comprises nucleotide monomers and activators. In these embodiments the computer program controls the activation of the orifices to dispense at least the nucleotide monomers and activator for the entire method until the array of biopolymers is prepared. In some embodiments the computer program controls the activation of the orifices to dispense at least the activator for the entire method until the array of biopolymers is prepared. In some embodiments the computer program controls the activation of the orifices to dispense at least the nucleotide monomers and activator for a portion of the method until the array of biopolymers is patterned.

### Specific Embodiments of Apparatus

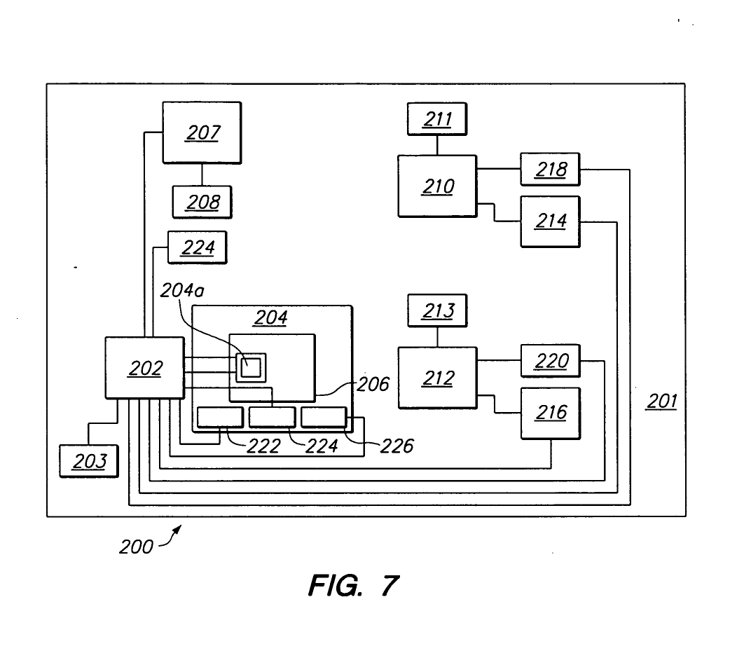

FIG. 7 depicts schematically an apparatus in accordance with embodiments of the present invention. Apparatus 200 comprises platform 201 on which the components of the apparatus are mounted. Apparatus 200 comprises main computer 202, with which various components of the apparatus are in communication. Video display 203 is in communication with computer 202. Apparatus 200 further comprises print chamber 204, which is controlled by main computer 202. The nature of print chamber 204 depends on the nature of the drop dispensing device and the like. Within print chamber 204 is drop dispensing device 204a and drop dispensing device moving mechanism 205a, which are adapted to move drop dispensing device 204a relative to a surface of a substrate on substrate mount 206 to bring the drop dispensing device into drop dispensing relationship with the surface of the substrate. Transfer robot 207 is also controlled by main computer 202 and comprises a robot arm 208 that moves a substrate to be printed from print chamber 204 to either first flow cell assembly 210 or second flow cell assembly 212. First flow cell assembly 210 is in communication with program logic controller 214, which is controlled by main computer 202, and second flow cell 212 is in communication with program logic controller 216, which is also controlled by main computer 202. First flow cell 210 assembly is in communication with fluid dispensing station 211 and flow sensor and level indicator 218, which are controlled by main computer 202, and second flow cell assembly 212 is in communication with fluid dispensing station 213 and flow sensor and level indicator 220, which are also controlled by main computer 202. Camera 222 is in communication with main computer 202.

Apparatus 200 also comprises loading station 224, which can be of any construction with regions that can retain small volumes of different fluids for loading into droplet dispensing device 204a. Loading station 224 may comprise a plurality of depots, from which liquids are to be transferred to drop dispensing device 204a. Loading station 204 is in fluid communication with drop dispensing device 204a. A motor system (not shown), controlled by computer 202, can be operated to move loading station 224 so that loading station 224 may be moved into position under drop dispensing device 204a and to load the dispensers with respective reagent fluids.

Apparatus 200 may optionally comprise a cleaning station or wash station 226. Cleaning station or wash station 226 may be employed, for example, to wipe or wash the surfaces of the dispensers and, optionally, subsequently dry the surfaces of the dispensers.

Apparatus 200 further comprises appropriate electrical and mechanical architecture and electrical connections, wiring and devices such as timers, clocks, and so forth for operating the various elements of the apparatus. Such architecture is familiar to those skilled in the art and will not be discussed in more detail herein.

### Use of Arrays

Arrays synthesized in accordance with embodiments of the present methods may be utilized in many diagnostic procedures in proteomics, genomics, and so forth.

For example, determining the nucleotide sequences and expression levels of nucleic acids (DNA and RNA) is critical to understanding the function and control of genes and their relationship, for example, to disease discovery and disease management. Analysis of genetic information plays a crucial role in biological experimentation. This has become especially true with regard to studies directed at understanding the fundamental genetic and environmental factors associated with disease and the effects of potential therapeutic agents on the cell. Such a determination permits the early detection of infectious organisms such as bacteria, viruses, etc.; genetic diseases such as sickle cell anemia; and various cancers. This paradigm shift has lead to an increasing need within the life science industries for more sensitive, more accurate and higher-throughput technologies for performing analysis on genetic material obtained from a variety of biological sources.

Unique or misexpressed nucleotide sequences in a polynucleotide can be detected by hybridization with a nucleotide multimer, or oligonucleotide, probe. Hybridization is based on complementary base pairing. When complementary single stranded nucleic acids are incubated together, the complementary base sequences bind to one another or pair to form double stranded hybrid molecules. These techniques rely upon the inherent ability of nucleic acids to form duplexes via hydrogen bonding according to Watson-Crick base-pairing rules. The ability of single stranded deoxyribonucleic acid (ssDNA) or ribonucleic acid (RNA) to form a hydrogen bonded structure with a complementary nucleic acid sequence has been employed as an analytical tool in molecular biology research. An oligonucleotide probe employed in the detection is selected with a nucleotide sequence complementary, usually exactly complementary, to the nucleotide sequence in the target nucleic acid. Following hybridization of the probe with the target nucleic acid, any oligonucleotide probe/nucleic acid hybrids that have formed are typically separated from unhybridized probe. The amount of oligonucleotide probe in either of the two separated media is then tested to provide a qualitative or quantitative measurement of the amount of target nucleic acid originally present.

Direct detection of labeled target nucleic acid hybridized to surface-bound polynucleotide probes is particularly advantageous if the surface contains a mosaic of different probes that are individually localized to discrete, and often known, areas of the surface. Such ordered arrays containing a large number of oligonucleotide probes have been developed as tools for high throughput analyses of genotype and gene expression. Oligonucleotides synthesized on a solid substrate recognize uniquely complementary nucleic acids by hybridization, and arrays can be designed to define specific target sequences, analyze gene expression patterns or identify specific allelic variations. The arrays may be used for conducting cell study, diagnosing disease, identifying gene expression, monitoring drug response, determination of viral load, identifying genetic polymorphisms, analyzing gene expression patterns or identifying specific allelic variations, and the like.

In one approach, cell matter is lysed, to release its DNA as fragments, which are then separated out by electrophoresis or other means, and then tagged with a fluorescent or other label. The resulting DNA mix is exposed to an array of oligonucleotide probes, whereupon selective binding to matching probe sites takes place. The array is then washed and examined or interrogated to determine the extent of hybridization reactions. Arrays of different chemical compounds or moieties or probe species provide methods of highly parallel detection, and hence improved speed and efficiency, in assays. Assuming that the different sequence polynucleotides were correctly deposited in accordance with the predetermined configuration, then the observed binding is indicative of the presence and/or concentration of one or more polynucleotide components of the sample.

Any suitable examining approach may be utilized. The nature of the examining device including a detector for examining the array for the results of one or more chemical reactions is dependent on the nature of the chemical reactions including any label employed for detection, such as fluorescent as mentioned above, chemiluminescent, calorimetric based on an attached enzyme, and the like. As mentioned above, the examining device may be a scanning device involving an imaging system or optical system. Other known examining devices may be employed. Such devices may involve the use of other optical techniques (for example, optical techniques for detecting chemiluminescent or electroluminescent labels) or electrical techniques (where each feature is provided with an electrode to detect hybridization at that feature in a manner disclosed in U.S. Pat. Nos. 6,221,583 and 6,251,685, and elsewhere). Other examining techniques include visual inspection techniques, non-light based methods, and so forth.

The signal referred to above may arise from any moiety that may be incorporated into the sample being analyzed for the purpose of detection. Often, a label is employed, which may be a member of a signal producing system. The label is capable of being detected directly or indirectly. In general, any reporter molecule that is detectable can be a label. Labels include, for example, (i) reporter molecules that can be detected directly by virtue of generating a signal, (ii) specific binding or reacting pair members that may be detected indirectly by subsequent binding or reacting to a cognate that contains a reporter molecule, (iii) mass tags detectable by mass spectrometry, (iv) oligonucleotide primers that can provide a template for amplification or ligation, (v) specific labeled nucleotide monomers which are incorporated into the target samples by enzymatic or chemical incorporation means, and (vi) a specific polynucleotide sequence or recognition sequence that can act as a ligand such as for a repressor protein, wherein in the latter two instances the oligonucleotide primer or repressor protein will have, or be capable of having, a reporter molecule and so forth. The reporter molecule can be a catalyst, such as an enzyme, a polynucleotide coding for a catalyst, promoter, dye, fluorescent molecule, chemiluminescent molecule, coenzyme, enzyme substrate, radioactive group, a small organic molecule, amplifiable polynucleotide sequence, a particle such as latex or carbon particle, metal sol, crystallite, liposome, cell, etc., which may or may not be further labeled with a dye, catalyst or other detectable group, a mass tag that alters the weight of the molecule to which it is conjugated for mass spectrometry purposes, and the like.

The signal may be produced by a signal producing system, which is a system that generates a signal that relates to the presence or amount of a target polynucleotide in a medium. The signal producing system may have one or more components, at least one component being the label. The signal producing system includes all of the reagents required to produce a measurable signal. The signal producing system provides a signal detectable by external means, by use of electromagnetic radiation, desirably by visual examination.

The arrays prepared as described above are particularly suitable for conducting hybridization reactions. Such reactions are carried out on an array comprising a plurality of features relating to the hybridization reactions. The array is exposed to liquid samples and to other reagents for carrying out the hybridization reactions. The substrate surface exposed to the sample is incubated under conditions suitable for hybridization reactions to occur.

After the appropriate period of time of contact between the liquid sample and the array, the contact is discontinued and various processing steps are performed. Following the processing step(s), the array is moved to an examining device as discussed above where the array is interrogated.

Results from the reading may be raw results (such as fluorescence intensity readings for each feature in one or more color channels) or may be processed results such as obtained by rejecting a reading for a feature that is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

When one item is indicated as being “remote” from another, this means that the two items are at least in different buildings and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

All publications and patent applications cited in this specification are herein incorporated by reference as if each individual publication or patent application were specifically and individually indicated to be incorporated by reference, except insofar as they may conflict with those of the present application (in which case the present application prevails). Methods recited herein may be carried out in any order of the recited events, which is logically possible, as well as the recited order of events.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for preparing an array of biopolymer features on a substrate by depositing drops of biopolymer forming reagents thereon, said method comprising:
(a) initiating multiple cycles of drop deposition of biopolymer forming reagents to feature locations on the surface of the substrate, wherein each cycle comprises at least one step of deposition of biopolymer monomers or biopolymer subunits wherein, for at least one of the biopolymer forming reagents and for at least a portion of the method, a dispensing protocol is employed, said dispensing protocol comprising dispensing drops of one or more biopolymer forming reagents wherein each biopolymer forming reagent is dispensed from a separate drop dispenser of a drop dispensing device and wherein each drop dispenser comprises multiple orifices and wherein multiple passes of the drop dispensing devices are employed for each cycle and wherein in each cycle drops of reagents are dispensed to feature locations from at least two different orifices for each reagent in at least two different passes of a drop dispensing device, and
(b) repeating the cycles to prepare the array of biopolymer features.

2. A method according to claim 1 wherein the two different orifices are on the same drop dispenser.

3. A method according to claim 1 wherein the two different orifices are on two different drop dispensers.

4. A method according to claim 1 wherein the biopolymer is selected from the group consisting of polynucleotides, polypeptides, polysaccharides and poly peptide-nucleic acid analogs.

5. A method according to claim 1 wherein the biopolymer forming reagents comprise polymer subunits selected from the group consisting of nucleotides and analogs thereof, amino acids and analogs thereof, and combinations thereof.

6. A method according to claim 1 wherein the biopolymer features are polynucleotide features and the biopolymer forming reagents comprises nucleotide monomers and activators.

7. A method according to claim 6 wherein at least the nucleotide monomers and activator are dispensed for the entire method until the array of biopolymers is prepared.

8. A method according to claim 6 wherein at least the activator is dispensed for the entire method until the array of biopolymers is prepared.

9. A method according to claim 6 wherein at least the nucleotide monomers and activator are dispensed for a portion of the method until the array of biopolymers is patterned.

10. A method for preparing an array of polynucleotide features on a substrate by depositing drops of polynucleotide forming reagents thereon, said method comprising:
(a) initiating multiple cycles of drop deposition of polynucleotide forming reagents to feature locations on the surface of the substrate, wherein each cycle comprises at least one step of deposition of polynucleotide monomers or polynucleotide subunits wherein, for at least one of the polynucleotide forming reagents and for at least a portion of the method, a dispensing protocol is employed, said dispensing protocol comprising dispensing drops of one or more polynucleotide forming reagents to a surface of a substrate in multiple cycles wherein each polynucleotide forming reagent is dispensed from a separate drop dispenser of a drop dispensing device and wherein each drop dispenser comprises multiple orifices and wherein multiple passes of the drop dispensing devices are employed for each cycle and wherein in each cycle drops of reagents are dispensed to feature locations from at least two different orifices for each reagent in at least two different passes of a drop dispensing device, and
(b) repeating the cycles to prepare the array of polynucleotide features.

11. A method according to claim 10 wherein the two different orifices are on the same drop dispenser.

12. A method according to claim 10 wherein the two different orifices are on two different drop dispensers.

13. A method according to claim 10 wherein the polynucleotide forming reagents comprise nucleotides and analogs thereof and combinations thereof.

14. A method according to claim 10 wherein the polynucleotide forming reagent is an activator.

15. A method according to claim 10 wherein the polynucleotide forming reagents comprise nucleotide monomers and an activator and at least the nucleotide monomers and activator are dispensed for the entire method until the array of biopolymers is prepared.

16. A method according to claim 10 wherein the polynucleotide forming reagents comprise nucleotide monomers and an activator and at least the activator is dispensed for the entire method until the array of biopolymers is prepared.

17. A method according to claim 10 wherein at least the nucleotide monomers and activator are dispensed for a portion of the method until the array of biopolymers is patterned.

18. An apparatus for preparing an array of biopolymer features on a surface of a substrate, said apparatus comprising:
(a) at least one drop dispensing device comprising one or more drop dispensers comprising multiple orifices,
(b) a mechanism for moving the drop dispensing device or a substrate relative to one another in one or more drop dispensing cycles to dispense biopolymer forming reagents to the surface of the substrate, and
(c) a computer comprising a computer program to control the movement of the mechanism and the activation of the orifices of a drop dispenser of the drop dispensing device in a protocol such that, for at least one of the biopolymer forming reagents and for at least a portion of the method, drops of reagents are dispensed to feature locations from at least two different orifices for each reagent in at least two different passes of a drop dispensing device.

19. An apparatus according to claim 18 wherein the computer program controls the activation of the orifices such that the two different orifices are on the same drop dispenser.

20. An apparatus according to claim 18 wherein the apparatus comprises at least two different drop dispensers for each reagent dispensed and wherein the computer program controls the activation of the orifices such that the at least two different orifices are on the at least two different drop dispensers.

21. An apparatus according to claim 18 comprising at least one drop dispenser for each different reagent to be dispensed.

22. An apparatus according to claim 18 comprising at least two drop dispensers for each different reagent to be dispensed.

23. An apparatus according to claim 18 wherein the biopolymer features are polynucleotide features and the biopolymer forming reagents comprises nucleotide monomers and activators and wherein the computer program controls the activation of the orifices to dispense at least the nucleotide monomers and activator for the entire method until the array of biopolymers is prepared.

24. An apparatus according to claim 18 wherein the biopolymer features are polynucleotide features and the biopolymer forming reagents comprises nucleotide monomers and activators and wherein the computer program controls the activation of the orifices to dispense at least the activator for the entire method until the array of biopolymers is prepared.

25. An apparatus according to claim 18 wherein the biopolymer features are polynucleotide features and the biopolymer forming reagents comprises nucleotide monomers and activators and wherein the computer program controls the activation of the orifices to dispense at least the nucleotide monomers and activator for a portion of the method until the array of biopolymers is patterned.
